(19) United States
(12) Patent Application Publication
(10) Pub. No.: US 2013/0202593 A1
(43) Pub. Date: Aug. 8, 2013

(54) INTRACELLULAR IMMUNITY

(71) Applicant: Medical Research Council, Swindon (GB)

(72) Inventors: Leo C. James, Cambridge (GB); Donna L. Mallery, Hertfordshire (GB); William A. McEwan, Cambridge (GB); Susanna R. Bidgood, Cambridge (GB)

(73) Assignee: MEDICAL RESEARCH COUNCIL, Swindon (GB)

(21) Appl. No.: 13/747,897

(22) Filed: Jan. 23, 2013

Related U.S. Application Data
(63) Continuation-in-part of application No. PCT/GB2011/001116, filed on Jul. 25, 2011.
(60) Provisional application No. 61/367,220, filed on Jul. 23, 2010.

(30) Foreign Application Priority Data
Jul. 23, 2010 (GB) 1012410.5

(51) Int. Cl. C07K 16/00; C07K 14/47

(52) U.S. Cl. CPC C07K 16/00; C07K 14/47
USPC 424/134.1; 530/387.3

(57) ABSTRACT

A compound which may comprise a ligand which binds, directly or indirectly, specifically to an antigen of a pathogen, provided that said ligand is not the PRYSPRY domain of TRIM21; and a RING domain and/or an inducer of TRIM21 expression.

### RELATED APPLICATIONS AND INCORPORATION BY REFERENCE

This application is a continuation-in-part application of international patent application Serial No. PCT/GB2011/001116 filed 25 Jul. 2011, which published as PCT Publication No. WO 2012/010855 on 26 Jan. 2012, which claims benefit of GB patent application Serial No. 1012410.5 and U.S. provisional patent application Ser. No. 61/367,220 filed 23 Jul. 2010.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention relates to compounds which are conjugates of a ligand specific for a pathogen, and a RING domain. In one embodiment, the RING domain is derived from a TRIM polypeptide, such as TRIM21.

### BACKGROUND OF THE INVENTION

Viruses and their hosts have been co-evolving for millions of years and this has given rise to a complex system of immunity traditionally divided into innate and adaptive responses¹. Innate immunity may comprise germ-line encoded receptors and effector mechanisms that recognise pathogen-associated molecular patterns, or PAMPs². The advantage of innate immunity is that it is fast and generic; however viruses are adept at avoiding recognition by inhibiting innate immunity or by changing their molecular patterns. In contrast, adaptive immunity can ‘cure’ a host of infection and provide protection against future infection. Unlike the PAMP receptors of innate immunity, adaptive immunity uses proteins such as antibodies to target pathogens. Antibodies are unique in the human body in that they evolve during the lifetime of an individual and can continue to target evolving pathogens³. The weakness of adaptive immunity is that it can take 1-2 weeks to reach full effectiveness. Furthermore, the dogma of antibody immunity for the last 100 years has been that antibodies only provide extracellular protection¹. It is thought that once a virus has entered the cytosol of a cell, antibodies are unable to prevent its infection.

Intracellular antibodies have been developed; for example, see Moutel S, Perez F., Med Sci (Paris). 2009 December; 25(12):1173-6; Stocks M., Curr Opin Chem. Biol. 2005 August; 9(4):359-65. However, results using intracellular antibodies, or intrabodies, have been mixed. In general, attempts to develop intracellular antibodies have focussed on single chain antibody fragments, such as scFvs and single domain antibodies, such as V_{HH} antibodies and dAbs.

Antibodies and immune sera have long been used for the treatment of pathogenic infections. Fore example, horse antiserum was used in the 1890s to treat tetanus and diphtheria. However, antisera are seen as foreign by the human immune system, which reacts by producing antibodies against them, especially on repeat doses. During most of the 20th C, the adverse effect of animal antibodies prompted the use of human antiserum from donors who had recovered from disease, typically for prophylaxis of respiratory and hepatitis B infections. following a reduction in the popularity of antibody therapy due to problems with toxicity, humanised and human antibodies have eliminated such concerns, and led to a return of such therapeutic approaches. See Casadevall et al., Nature Reviews Microbiology 2, 695-703 (September 2004), for a review. Diseases which have been targeted using antibody therapy include anthrax, whooping cough, tetanus, botulism, cryptococcosis, cryptosporidiosis, enterovirus gastrointestinal-tract infections, group a streptococcal infections, necrotizing fasciitis, hepatitis B, measles, tuberculosis, meningitis, aplastic anaemia, rabies, RSV infection, pneumonia, shingles, chickenpox and pneumonia due to VZV, and smallpox. Despite these developments, however, antibody therapy is considered only when no other suitable therapies are available, requiring high doses of antibody and producing unpredictable results.

The effectiveness of antibodies against pathogens is understood to be at least partly dependent on the Fc portion of the antibody, which is responsible for mediating the effects of complement. Therefore, antibody fragments have not been generally proposed for antiviral therapy, despite their advantages of small size and lower cost of production.

The primary therapy for viral diseases remains vaccination, which is a prophylactic approach. It is believed that viral antigens, processed by antigen-presenting cells such as dendritic cells, are presented to the immune system and induce naïve T-cells to differentiate into memory and effector T-cells. Memory T-cells are responsible for the more aggressive and immediate immune response to a secondary infection, mediating the benefits of vaccination. For a review, see Kaech et al., Nature Reviews Immunology, volume 2, April 2002, 251.

Another immunologically-based approach to the therapy of infections disease is the use of cytokines, including inteferons. Interferon was first proposed for the treatment of cancer and multiple sclerosis, as well as viral infections. It has been licensed for the treatment of hepatitis C since 1998. Moreover, low dose oral or intranasal interferon is administered for the treatment of colds and flu, especially in eastern Europe. However, the mechanism of its action is not known, since the doses used are believed to be lower than the doses at which an antiviral effect could be observed. O'Brien et al., J Gen Virol. 2009 April; 90(Pt 4):874-82, used interferon as an adjuvant to an adenovirus-delivered vaccine against VEEV; they observed a decrease in protection against the virus, but an increase in the immune response to the viral vector.

Citation or identification of any document in this application is not an admission that such document is available as prior art to the present invention.

### SUMMARY OF THE INVENTION

Recently, Applicants described an intracellular cytosolic protein called TRIM21 that is capable of binding to an invariant region of antibody molecules via its PRYSPRY domain⁴. Applicants found this activity to be structurally, thermodynamically and kinetically conserved across mammals⁵. Hypotheses for the function of TRIM21 have been suggested, including its involvement in apoptosis and a role in directing of unfolded IgG made in B-cells to the proteasome.

Antibodies are extracellular proteins, as are all known mammalian IgG receptors (with the exception of FcRn, which is intracellular but not cytosolic). It therefore seemed incongruous to us that TRIM21 should be a universally conserved intracellular protein, and yet be a high affinity, highly specific IgG receptor. Applicants hypothesized that perhaps current understanding of antibody immunity is incomplete and that there is a ‘missing’ system of immunity taking place inside cells, mediated by TRIM21. Data presented herein demonstrates the existence of this missing immune system and its operation in preventing infection by two unrelated viruses—dsDNA Adenovirus and ssRNA Coxsackie virus.

In a first aspect of the invention, therefore, there is provided a compound which may comprise:
(a) a ligand which binds, directly or indirectly, specifically to an antigen of a pathogen, provided that said ligand is not the PRYSPRY domain of TRIM21; and
(b) a RING domain and/or an inducer of TRIM21 expression.

Applicants have shown that TRIM21 is a high-affinity ligand for immunoglobulins. the RING domain of TRIM 21 is an E3 ligase, which is ubiquitinated and directs the immunoglobulins, together with bound antigens, to the proteasome.

In accordance with the present invention, at least a RING domain, such as the RING domain of a TRIM polypeptide, can be bound to a ligand for an antigen. Such a ligand preferably binds directly to the antigen, and may comprise at least part of an immunoglobulin molecule; however, other ligands may be used, including peptides, peptide and nucleic acid-based aptamers, naturally-occurring ligands, receptors, and binding fragments thereof.

In another embodiment, the ligand binds indirectly to the antigen. For example, the ligand may bind to immunoglobulins non-specifically, such as to the Fc portion of an immunoglobulin. In such an embodiment, the ligand is not the TRIM21 PRYSPRY domain. Exemplary ligands include Protein A, Protein G, Protein L, peptides, for instance peptides which recognise immunoglobulin Fc regions, anti-Fc antibodies and fragments thereof, and the like. The target specificity is provided, in this case, by an antibody or antibody fragment which is specific for the antigen of the pathogen. This antibody may be coadministered with the compound of the invention, or may be naturally occurring.

In the context of the present invention, the term “ligand” is used to refer to either half of a binding pair.

Where the ligand is an immunoglobulin, it can be any immunoglobulin molecule, for example an immunoglobulin molecule selected from the group consisting of an IgG, IgA, IgM, IgE, IgD, F(ab′)₂, Fab, Fv, scFv, dAb, V_{HH}, IgNAR, a TCR, and multivalent combinations thereof. Multivalent antibodies include, for instance, bivalent antibodies and antibody fragments, bispecific antibodies and antibody fragments, trivalent versions thereof, and proprietary formats such as diabodies. Single domain antibodies, such as dAbs and V_{HH} antibodies, are particularly suitable for combining to form multivalent and/or multispecific molecules.

Where the ligand is an antibody, the antibody molecule may comprise at least one of a V_{H} domain and a V_{L} domain, or the equivalent thereof.

In one embodiment, the TRIM polypeptide is selected from the group consisting of TRIM5a, TRIM19, TRIM21 and TRIM28. Although TRIM21 is preferred due to its antibody-binding properties, if the polypeptide, or a domain thereof, is bound to the antigen itself, or a ligand specific for the antigen, the antibody-binding ability is no longer required. In such an instance, the RING domain from a TRIM polypeptide other than TRIM21 can be used, to the same effect. Advantageously, another domain, such as the B-box domain or the coiled coil domain, can also be added. The coiled coil domain is responsible for TRIM21 dimerisation.

Preferably, the RING domain is present in two or more copies on the compound according to the invention. Dimerisation of TRIM21 occurs through its coiled coil domain, and assists in the targeting of the protein to the proteasome through E3-mediated ligation of ubiquitin.

In one embodiment, the compound of the invention may comprise a substantially intact TRIM polypeptide, wherein the PRYSPRY (B30.2) domain has been replaced with an antigen or an antigen-specific ligand. For example, it can be replaced with an antibody, which may comprise at least one of a V_{H} domain and a V_{L} domain.

In a further embodiment, the compound of the invention may comprise an inducer of TRIM expression instead of, or as well as, a TRIM domain. TRIM21 expression is upregulated by interferon, so the inducer of TRIM expression is advantageously interferon or an interferon inducer. A variety of interferon inducers, including bacterial polysaccharides and nucleoside analogues such as poly I:C, are known in the art.

Interferon inducers can act intracelluarly, or at the cell surface. Where the interferon inducer acts at the cell surface, at least a proportion of the compound which is administered to a subject will be retained on the cell surface, bound to the interferon inducer receptor. In an advantageous embodiment, the interferon inducer may be bound to the compound by a labile linkage, for example a linkage with a limited half-life under physiological conditions. For example, the half life would be sufficient for the ligand to bid to the pathogen and to the interferon receptor, but not significantly longer.

In a second aspect, there is provided method for treating a pathogenic infection, which may comprise administering to the subject a compound according to the first aspect of the invention.

Similarly, there is provided the use of a compound according to the first aspect of the invention, for inducing an immune response in a subject.

In a third aspect, the invention provides a method for treating an infection in a subject, which may comprise co-administering to the subject an antibody specific for an antigen of a pathogen causing said infection, and a polypeptide which may comprise a ligand which binds to said antibody and a RING domain.

Similarly, there is provided the use of an antibody specific for an antigen of a pathogen causing an infection in a subject, and a polypeptide which may comprise a ligand which binds to said antibody and a RING domain, for the treatment of said infection.

Applicants have demonstrated that treatment of cells with a virus-specific antibody and wild-type or modified TRIM21 leads to inhibition of viral infectivity, even in cells in which endogenous TRIM21 has been knocked down. Accordingly, the coadministration of TRIM21 can be used to enhance antiviral therapy used for the treatment of an infectious disease.

In a fourth aspect, there is provided a method for treating an infection in a subject suffering from such an infection, which may comprise administering to the subject a therapeutically effective amount of a polypeptide which may comprise a ligand which binds, indirectly, to an antigen of a pathogen and a RING domain.

Similarly, there is provided the use of a polypeptide which may comprise a ligand which binds, indirectly, to an antigen of a pathogen and a RING domain for the treatment of an infectious disease in a subject.

Preferably, the polypeptide which may comprise the PRYSPRY domain of TRIM21 and a RING domain may comprise further domains of TRIM polypeptides, such as from TRIM21. In one embodiment, the polypeptide may comprise a coiled coil domain and/or a B-box domain. In one embodiment, the polypeptide is TRIM21, preferably human TRIM21.

TRIM21 has not previously been proposed to possess anti-infective properties. However, as shown herein, it binds with very high affinity to the Fc receptor of IgG and IgM, and directs the antibody plus any bound antigen to the proteasome. Exogenous TRIM21, therefore, potentiates an endogenous antibody response to a pathogen.

Applicants' results reveal that there is a missing system of intracellular immunity through which antibodies mediate the neutralisation of virus inside the cytosol of infected cells. This intracellular system combines features traditionally associated exclusively with either adaptive or innate immunity. Pathogen targeting is provided by adaptive immunity in the form of antibodies whereas neutralisation is provided by an intracellular receptor (TRIM21) and innate degradation pathway. TRIM21 is distinct from other antibody effector mechanisms, which are systemic and based on immune surveillance. TRIM21 is expressed in most cells and not just professional immune cells, which means that every infection event is an opportunity for neutralisation. Encapsulating immunity within host cells may be crucial to inhibiting viral spread. Finally, TRIM21 utilises both IgM and IgG suggesting that it operates alongside both innate immunity during the early stages of infection and adaptive immunity to provide long-term protection.

TRIM21 may have been contributing to many antibody neutralisation experiments over the last 100 years. Indeed, as Applicants see that TRIM21 mediates a potent antibody neutralisation of adenovirus it will be important to reassess whether the antibody neutralisation of other viruses is caused by a block to entry or is TRIM21-dependent. This may be an important consideration in vaccine design, as effective vaccines may need to stimulate TRIM21 immunity. Applicants suggest that a good predictor of TRIM21 involvement in the antibody neutralisation of other viruses will be a synergistic relationship between interferon and antibody. Indeed unexplained synergy between interferon and antibody has been reported for herpes simplex virus⁸, enterovirus 70⁸ and sindbis virus⁹. TRIM21 may also contribute to viral neutralisation in experiments where no antibody is added since the calf serum used in routine tissue culture contains a repertoire of antibodies of potentially cross-reactive specificity.

The existence of a TRIM21/antibody intracellular immune response may help to resolve several unexplained observations in viral infection. It has been reported that antibody neutralisation of both poliovirus¹⁰ and respiratory syncytial virus¹¹ occurs even when viruses are allowed to pre-adhere to target cells. It has also been observed that a single IgG is sufficient to mediate neutralisation of poliovirus¹² and adenovirus¹³ and only 5-6 IgG molecules are required for rhinovirus¹⁴. Finally, there are numerous reports of intact antibodies being far more effective than their proteolysed fragments, even than Fab2 that maintain bivalent antigen binding. For instance, Fab2 fragments have been shown to be less effective than intact IgG in the neutralisation of Yellow fever virus¹⁵, HSV¹⁶ and Influenza¹⁷, suggesting an Fc domain effector function for efficient neutralisation. TRIM21-mediated degradation may explain all these phenomena.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE FIGURES

The following detailed description, given by way of example, but not intended to limit the invention solely to the specific embodiments described, may best be understood in conjunction with the accompanying drawings.

FIG. 1: TRIM21 mediates intracellular antibody neutralisation. (A) Confocal microscopy images of adenovirus infected HeLa cells. Adenovirus pre-coated in antibody and detected after infection with an Alexa-fluor546 secondary (red) can be seen inside cells. Endogenous TRIM21 localisation is shown in green and DAPI stained nucleus in blue. A merge of these channels shows localisation of TRIM21 to antibody-coated virions. The images are a Z-projection and the scale bars are 10 μm and 2 μm in the zoomed box. (B) Cells treated with IFNα, TRIM21 siRNA (KD), siRNA control (HeLa) or IFNα & TRIM21 siRNA (IFNα KD) were infected with GFP adenovirus at different polyclonal antibody concentrations. The level of infection was determined by measuring the percentage of GFP positive cells and for each condition normalised to the levels in the absence of antibody. Adenovirus infection is reduced by 2-logs in cells expressing the highest levels of TRIM21. (C) Western blot of TRIM21 protein levels in each condition. (D) Infection of treated cells with coxsackievirus in the presence of increasing concentrations of human serum IgG. IFNα and antibody operate synergistically to neutralise virus. This effect is reversed by specifically knocking-down TRIM21.

FIG. 2: TRIM21 neutralises infection independent of cell-type and antibody. (A) Neutralisation of TRIM21 is reversed by knockdown, independent of siRNA sequence or siRNA vs shRNA, in the presence of antibody. (B) TRIM21 neutralises adenovirus infection in three different cell lines. Neutralisation is enhanced by IFNα and reversed by knockdown (KD). (C) TRIM21 neutralises adenovirus infection when using different polyclonals or an anti-hexon monoclonal IgG. (D) Entry neutralisation is minimal in comparison with TRIM21-mediated neutralisation. Antibody-dependent TRIM21 neutralisation of virus requires the presence of the Fc domain. Fab2 fragments are bivalent and have the same potential for entry-neutralisation as intact IgG yet they show a limited affect on infection. This is confirmed by knockdown of TRIM21, which reverses IgG neutralisation. (E) TRIM21 binds to serum IgM. Binding of IgM to TRIM21 was measured as a change in fluorescence anisotropy and fit to a standard quadratic expression (Materials & Methods) to give an affinity of 16.8 μM±1.5 μM. (F) Serum IgM antibodies can also be used by TRIM21 to neutralise virus. Knockdown of TRIM21 (KD) reverses this effect and IFNα increases it. Error bars in all panels were calculated from triplicate experiments. (G) IgA antibodies can moreover be used by TRIM21 to neutralise virus. Knockdown of TRIM21 using siRNA (siTRIM21) reverses this effect and IFNα increases it. Control siRNA (siControl) has no effect.

FIG. 3: Mechanism of TRIM21 neutralisation. (A) SEC MALS chromatograms of TRIM21 (black), IgG (light gray) and TRIM21 in complex with IgG (dark gray). The continuous traces represent the RI signal (left-hand axis) and are an indication of protein concentration. The short horizontal lines represent the calculated mass at each sampling interval (1s) within each peak (right-hand axis). Analysis indicates that TRIM21 is dimeric with a mass of 107 kDa, that IgG has a mass of 154 kDa, and that TRIM21:IgG complex yields a peak corresponding to free IgG and a 1:1 complex with mass ˜280 kDa. (B&C) Steady-state fluorescence titration of IgG with full-length TRIM21 (left-hand) and ΔRING-ΔBOX TRIM21 (right-hand). Titrations were fit to a standard quadratic expression (Materials & Methods) to give an affinity of full-length TRIM21 to antibody of 0.6±0.1 nM (B) and 0.9±0.2 nM for ΔRING-ΔBOX TRIM21 (C). (D) TRIM21 neutralisation is reversed by the proteasome inhibitor MG132 but not the autophagy inhibitor 3-MA. Error bars were calculated from triplicate experiments. (E) Direct correlation between MG132 concentration and reversal of TRIM21 neutralisation. MG132 only reverses neutralisation in the presence of antibody. (F) Proteasomal degradation, TRIM21 and antibody are necessary factors in the same pathway of viral neutralisation. For example, knockdown of TRIM21 obviates the MG132 affect.

FIG. 4: TRIM21 E3 ubiquitin ligase function is essential for viral neutralisation (A) Recombinant full-length TRIM21 neutralises virus but TRIM21 lacking the RING and B Box domains does not. (B) TRIM21 is an active E3 ligase but deletion of RING and B Box domains prevents autoubiquitination. (C) TRIM21 does not directly ubiquitinate hexon or its associated antibody during infection. (D) Confocal microscopy Z-projection showing HeLa cells infected with antibody-coated adenovirus. TRIM21 co-localised virions are positive for ubiquitin. (E) Western blots of hexon, antibody and TRIM21 protein levels 1-6 hours post-infection. Adenovirus hexon protein and antibody are rapidly degraded in a TRIM21-dependent manner. Addition of MG132 partially rescues degradation. Degradation does not significantly affect the cellular pool of TRIM21.

FIG. 5: Intracellular antibody-coated beads recruit TRIM21 and are ubiquitinated. Streptavidin-conjugated latex beads are coated with anti-streptavidin antibody and transfected into cells. Intracellular beads are recognised by TRIM21 and colocalise with ubiquitin. The scale bars represent 10 μm and 5 μm in the zoomed box.

FIG. 6: TRIM21 provides very potent protection against viral infection. Embryonic fibroblast cells were prepared from either wild-type or TRIM21 KO C57BL/6 mice and challenged with GFP-adenovirus in the presence of 9C12, a monoclonal anti-hexon antibody.

FIG. 7: Schematic representation of the synthesis of various loop peptide constructs.

FIG. 8: Infection of wild type mice with 4×10⁵ pfu resulted in 75% mortality rate. For LD50 determinations, six-week-old C57BL6 mice were infected by intraperitoneal (i.p.) injection (four animals per dose) of 10-fold serially diluted doses of MAV-1 in 100 ul of PBS and observed up to twice daily for morbidity and mortality.

### DESCRIPTION OF THE INVENTION

Unless otherwise stated, all technical and scientific terms used herein have the same meanings as commonly understood by one of ordinary skill in the art to which this invention belongs. Any methods and materials similar or equivalent to those described herein can be used in the practice or testing of the present invention. Methods, devices, and materials suitable for such uses are now described. All publications cited herein are incorporated herein by reference in their entirety for the purpose of describing and disclosing the methodologies, reagents, and tools reported in the publications that might be used in connection with the invention.

The practice of the present invention employs, unless otherwise indicated, conventional methods of chemistry, biochemistry, molecular biology, cell biology, genetics, immunology and pharmacology, known to those of skill of the art. Such techniques are explained fully in the literature. See, e.g., Gennaro, A. R., ed. (1990) Remington's Pharmaceutical Sciences, 18th ed., Mack Publishing Co.; Hardman, J. G., Limbird, L. E., and Gilman, A. G., eds. (2001) The Pharmacological Basis of Therapeutics, 10th ed., McGraw-Hill Co.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series), 2nd ed., Springer Verlag.

In the context of the present invention, administration is performed by standard techniques of cell culture, depending on the reagent, compound or gene construct to be administered. For instance, administration may take place by addition to a cell culture medium, introduction into cells by precipitation with calcium phosphate, by electroporation, by viral transduction or by other means. If the method of the invention employs a non-human mammal as the test system, the mammal may be transgenic and express the necessary reagents in its endogenous cells.

An antigen, in the context of the present invention, is a molecule which can be recognised by a ligand and which possesses an epitope specific for a pathogen. Typically, an antigen is an antigenic determinant of a pathogen, such as a virus or a bacterium, and is exposed to binding by ligands such as antibodies under physiological conditions. Preferred antigens comprise epitopes targeted by known neutralising antibodies or vaccines specific for a pathogen.

A pathogen may be any foreign body, such as an organism, for example a bacterium or a protozoan, or a virus, which can infect a subject. Advantageously, the pathogen is a virus. Viruses may be enveloped or non-enveloped. In one embodiment, the pathogen is a non-enveloped virus.

A ligand which binds directly to an antigen is a ligand which is capable of binding specifically to an antigen under physiological conditions. As used herein, the term “ligand” can refer to either part of a specific binding pair; for instance, it can refer to the antibody or the antigen in an antibody-antigen pair. Antibodies are preferred ligands, and may be complete antibodies or antibody fragments as are known in the art, which may comprise for example IgG, IgA, IgM, IgE, IgD, F(ab)₂, Fab, Fv, scFv, dAb, V_{HH}, IgNAR, a modified TCR, and multivalent combinations thereof. Ligands may also be binding molecules based on alternative non-immunoglobulin scaffolds, peptide aptamers, nucleic acid aptamers, structured polypeptides which may comprise polypeptide loops subtended on a non-peptide backbone, natural receptors or domains thereof.

A ligand which binds indirectly to an antigen is a Iigand which binds to the antigen via a second ligand. For instance, it is a ligand which binds to an antibody. The ligand binds the antibody in a manner independent of the binding specificity of the antibody; for instance, it can bind the Fc region. In one embodiment, the ligand is selected from the group which may comprise Protein G, protein A, Protein L, the PRYSPRY domain of TRIM21, an anti-immunoglobulin antibody, and peptides which specifically recognise antibodies, for example in the Fc region.

The PRYSPRY domain of TRIM21 is comprised of the PRY and SPRY regions, respectively at positions 286-337 and 339-465 of the human TRIM21 amino acid sequence, as set forth in SEQ ID No. 1.

The RING domain is of human TRIM21 between amino acids 15 and 58 of the human TRIM21 amino acid sequence, as set forth in SEQ ID No. 1.

The BBOX domain is of human TRIM21 between amino acids 91 and 128 of the human TRIM21 amino acid sequence, as set forth in SEQ ID No. 1.

The Coiled Coil domain is of human TRIM21 between amino acids 128 and 238 of the human TRIM21 amino acid sequence, as set forth in SEQ ID No. 1.

The term “immunoglobulin” refers to a family of polypeptides which retain the immunoglobulin fold characteristic of antibody molecules, which contains two beta sheets and, usually, a conserved disulphide bond. Members of the immunoglobulin superfamily are involved in many aspects of cellular and non-cellular interactions in vivo, including widespread roles in the immune system (for example, antibodies, T-cell receptor molecules and the like), involvement in cell adhesion (for example the ICAM molecules) and intracellular signalling (for example, receptor molecules, such as the PDGF receptor). The present invention is applicable to all immunoglobulin superfamily molecules which possess binding domains. Preferably, the present invention relates to antibodies.

An antigen is specific to a pathogen if targeting the antigen results in substantially exclusive targeting of the pathogen under physiological conditions.

The variable domains of the heavy and light chains of immunoglobulins, and the equivalents in other proteins such as the alpha and beta chains of T-cell receptors, are responsible for determining antigen binding specificity. V_{H} and V_{L} domains are capable of binding antigen independently, as in V_{H} and V_{L} dAbs. References to V_{H} and V_{L} domains include modified versions of V_{H} and V_{L} domains, whether synthetic or naturally occurring. For example, naturally occurring V_{H} variants include camelid V_{HH} domains, and the heavy chain immunoglobulins IgNAR of cartilaginous fish.

A TRIM polypeptide is a member of the tripartite motif (TRIM) family of proteins, which may comprise 70 members in the human genome, including TRIM21 (Ro52). TRIM proteins are involved in diverse cellular processes, including cell proliferation, differentiation, development, oncogenesis, and apoptosis. TRIM proteins are multidomain, so-called because of their concerved N-terminal RBCC domains: a RING finger encoding E3 ubiquitin ligase activity, a B-box, and a coiled-coil domain mediating oligomerization. The C-terminal PRYSPRY or B30.2 domain commonly determines function of different TRIM polypeptides, by acting as a targeting module. See Nisole et al., Nature Reviews Microbiology 3, 799-808 (October 2005). The RING domain is defined by a regular arrangement of cysteine and histidine residues that coordinate two atoms of zinc, and is found in a large variety of proteins. It is characterised by the structure C-X2-C-X(9-39)-C-X(1-3)-H-X(2-3)-(N/C/H)-X2-C-X(4-48)C-X2-C, and associated with a B-box domain in TRIM polypeptides. See Freemont, Curr Biol. 2000 Jan. 27; 10(2):R84-7.

A domain is a folded protein structure which retains its tertiary structure independently of the rest of the protein. Generally, domains are responsible for discrete functional properties of proteins, and in many cases may be added, removed or transferred to other proteins without loss of function of the remainder of the protein and/or of the domain. The RING, B-box, Coiled Coil and PRYSPRY domains of TRIM polypeptides are examples thereof. By antibody variable domain is meant a folded polypeptide domain which may comprise sequences characteristic of antibody variable domains. It therefore includes complete antibody variable domains and modified variable domains, for example in which one or more loops have been replaced by sequences which are not characteristic of antibody variable domains, or antibody variable domains which have been truncated or comprise N- or C-terminal extensions, as well as folded fragments of variable domains which retain at least in part the binding activity and specificity of the full-length domain.

An inducer of TRIM expression is an agent which increases intracellular levels of a desired TRIM polypeptide. Preferably, the polypeptide is TRIM21. Type I interferon in an inducer of TRIM21 expression.

As referred to herein, coadministration is the simultaneous, simultaneous separate or sequential administration of two agents, such that they are effective at the same time at the site of interest. In the context of the coadministration of an antibody and a TRIM21 polypeptide, therefore, the two agents should be administered such that the antibody is bound by the TRIM21 polypeptide prior to internalisation by the cell. Thus, the antibody and the TRIM21 polypeptide can be admixed prior to administration, or separately administered such that they are present in the circulation at the same time.

Antibodies target pathogens before they infect cells. Applicants show herein that upon infection these antibodies remain bound to pathogens and direct an intracellular immune response that is present inside every cell. Applicants demonstrate that each cell posses a cytoplasmic IgG receptor, TRIM21, which binds to antibodies with a higher affinity than any other IgG receptor in the human body. This enables TRIM21 to rapidly recruit to intracellular antibody-bound virus and target it for degradation in the proteasome via its E3 ubiquitin ligase activity. At physiological antibody concentrations, TRIM21 completely neutralises viral infection. These findings represent an unprecedented system of broad-spectrum immunity, revealing that the protection mediated by antibodies does not end at the cell membrane but continues inside the cell to provide a last line of defence against infection.

The PRYSPRY domain of TRIM21 is responsible for antibody binding, and in this sense TRIM21 appears to be unique in the TRIM polypeptide family. However, the TRIM domains which is responsible for proteasome targeting, the RING domain, is not specific to TRIM21; rather, it is common to proteins including the TRIM family.

Induction of TRIM21 expression in cells is dependent on interferon, which is subject to delay and to interference by viral mechanisms. Therefore, the present invention provides antigen-specific ligands which are fused to a RING domain, such that when the pathogen is internalised by the cell, ligands bound to the pathogen immediately direct it to the proteasome for degradation. This effectively allows the cell to cure itself of the pathogen infection.

Any ligand which can bind to a pathogen-associated antigen under physiological conditions, and be internalized by a cell, is suitable for use in the present invention. The natural immune system uses antibodies as ligands for pathogens, and antibodies or antibody fragments are ideal for use in the present invention. Other possibilities include binding domains from other receptors, as well as engineered peptides and nucleic acids.

References herein to antigen- or pathogen-specific antibodies, antigen- or pathogen-binding antibodies and antibodies specific for an antigen or pathogen are coterminous and refer to antibodies, or binding fragments derived from antibodies, which bind to antigens which are present on a pathogen in a specific manner and substantially do not cross-react with other molecules present in the circulation or the tissues.

An “antibody” as used herein includes but is not limited to, polyclonal, monoclonal, recombinant, chimeric, complementarity determining region (CDR)-grafted, single chain, bi-specific, Fab fragments and fragments produced by a Fab expression library. Such fragments include fragments of whole antibodies which retain their binding activity for the desired antigen, Fv, F(ab′), F(ab′)2 fragments, and F(v) or V_{H} antibody fragments as well as fusion proteins and other synthetic proteins which comprise the antigen-binding site of the antibody. Furthermore, the antibodies and fragments thereof may be human or humanized antibodies, as described in further detail below.

Antibodies and fragments also encompass antibody variants and fragments thereof. Variants include peptides and polypeptides which may comprise one or more amino acid sequence substitutions, deletions, and/or additions that have the same or substantially the same affinity and specificity of epitope binding as the antigen-specific antibody or fragments thereof.

The deletions, insertions or substitutions of amino acid residues may produce a silent change and result in a functionally equivalent substance. Deliberate amino acid substitutions may be made on the basis of similarity in polarity, charge, solubility, hydrophobicity, hydrophilicity, and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Homologous substitution (substitution and replacement are both used herein to mean the interchange of an existing amino acid residue, with an alternative residue) may occur i.e. like-for-like substitution such as basic for basic, acidic for acidic, polar for polar etc. Non homologous substitution may also occur i.e. from one class of residue to another or alternatively involving the inclusion of unnatural amino acids—such as ornithine (hereinafter referred to as Z), diaminobutyric acid ornithine (hereinafter referred to as B), norleucine ornithine (hereinafter referred to as O), pyriylalanine, thienylalanine, naphthylalanine and phen ylglycine.

Thus, variants may include peptides and polypeptides which may comprise one or more amino acid sequence substitutions, deletions, and/or additions to the antigen specific antibodies and fragments thereof wherein such substitutions, deletions and/or additions do not cause substantial changes in affinity and specificity of epitope binding. Variants of the antibodies or fragments thereof may have changes in light and/or heavy chain amino acid sequences that are naturally occurring or are introduced by in vitro engineering of native sequences using recombinant DNA techniques. Naturally occurring variants include “somatic” variants which are generated in vivo in the corresponding germ line nucleotide sequences during the generation of an antibody response to a foreign antigen.

Variants of antibodies and binding fragments may also be prepared by mutagenesis techniques. For example, amino acid changes may be introduced at random throughout an antibody coding region and the resulting variants may be screened for binding affinity for the target antigen, or for another property. Alternatively, amino acid changes may be introduced into selected regions of the antibody, such as in the light and/or heavy chain CDRs, and/or in the framework regions, and the resulting antibodies may be screened for binding to the target antigen or some other activity. Amino acid changes encompass one or more amino acid substitutions in a CDR, ranging from a single amino acid difference to the introduction of multiple permutations of amino acids within a given CDR. Also encompassed are variants generated by insertion of amino acids to increase the size of a CDR.

The antigen-binding antibodies and fragments thereof may be humanized or human engineered antibodies. As used herein, “a humanized antibody”, or antigen binding fragment thereof, is a recombinant polypeptide that may comprise a portion of an antigen binding site from a non-human antibody and a portion of the framework and/or constant regions of a human antibody. A human engineered antibody or antibody fragment is a non-human (e.g., mouse) antibody that has been engineered by modifying (e.g., deleting, inserting, or substituting) amino acids at specific positions so as to reduce or eliminate any detectable immunogenicity of the modified antibody in a human.

Humanized antibodies include chimeric antibodies and CDR-grafted antibodies. Chimeric antibodies are antibodies that include a non-human antibody variable region linked to a human constant region. Thus, in chimeric antibodies, the variable region is mostly non-human, and the constant region is human. Chimeric antibodies and methods for making them are described in, for example, Proc. Natl. Acad. Sci. USA, 81: 6841-6855 (1984). Although, they can be less immunogenic than a mouse monoclonal antibody, administrations of chimeric antibodies have been associated with human immune responses (HAMA) to the non-human portion of the antibodies.

CDR-grafted antibodies are antibodies that include the CDRs from a non-human “donor” antibody linked to the framework region from a human “recipient” antibody. Methods that can be used to produce humanized antibodies also are described in, for example, U.S. Pat. Nos. 5,721,367 and 6,180,377.

“Veneered antibodies” are non-human or humanized (e.g., chimeric or CDR-grafted antibodies) antibodies that have been engineered to replace certain solvent-exposed amino acid residues so as to reduce their immunogenicity or enhance their function. Veneering of a chimeric antibody may comprise identifying solvent-exposed residues in the non-human framework region of a chimeric antibody and replacing at least one of them with the corresponding surface residues from a human framework region. Veneering can be accomplished by any suitable engineering technique.

Further details on antibodies, humanized antibodies, human engineered antibodies, and methods for their preparation can be found in Antibody Engineering, Springer, New York, N.Y., 2001.

Examples of humanized or human engineered antibodies are IgG, IgM, IgE, IgA, and IgID antibodies. The antibodies may be of any class (IgG, IgA, IgM, IgE, IgD, etc.) or isotype and can comprise a kappa or lambda light chain. For example, a human antibody may comprise an IgG heavy chain or defined fragment, such as at least one of isotypes, IgG1, IgG2, IgG3 or IgG4. As a further example, the antibodies or fragments thereof can comprise an IgG1 heavy chain and a kappa or lambda light chain.

The antigen specific antibodies and fragments thereof may be human antibodies—such as antibodies which bind the antigen and are encoded by nucleic acid sequences which may be naturally occurring somatic variants of human germline immunoglobulin nucleic acid sequence, and fragments, synthetic variants, derivatives and fusions thereof. Such antibodies may be produced by any method known in the art, such as through the use of transgenic mammals (such as transgenic mice) in which the native immunoglobulins have been replaced with human V-genes in the mammal chromosome.

Human antibodies to target a desired antigen can also be produced using transgenic animals that have no endogenous immunoglobulin production and are engineered to contain human immunoglobulin loci, as described in WO 98/24893 and WO 91/00906.

Human antibodies may also be generated through the in vitro screening of antibody display libraries (J. Mol. Biol. (1991) 227: 381). Various antibody-containing phage display libraries have been described and may be readily prepared. Libraries may contain a diversity of human antibody sequences, such as human Fab, Fv, and scFv fragments, that may be screened against an appropriate target. Phage display libraries may comprise peptides or proteins other than antibodies which may be screened to identify agents capable of selective binding to the desired antigen.

Phage-display processes mimic immune selection through the display of antibody repertoires on the surface of filamentous bacteriophage, and subsequent selection of phage by their binding to an antigen of choice. One such method is described in WO 99/10494. Antigen-specific antibodies can be isolated by screening of a recombinant combinatorial antibody library, preferably a scFv phage display library, prepared using human V_{L} and V_{H} cDNAs prepared from mRNA derived from human lymphocytes. Methodologies for preparing and screening such libraries are known in the art. There are commercially available kits for generating phage display libraries.

As used herein, the term “antibody fragments” refers to portions of an intact full length antibody—such as an antigen binding or variable region of the intact antibody. Examples of antibody fragments include Fab, Fab′, F(ab′)₂, and Fv fragments; diabodies; linear antibodies; single-chain antibody molecules (e.g., scFv); multispecific antibody fragments such as bispecific, trispecific, and multispecific antibodies (e.g., diabodies, triabodies, tetrabodies); binding-domain immunoglobulin fusion proteins; camelized antibodies; minibodies; chelating recombinant antibodies; tribodies or bibodies; intrabodies; nanobodies; small modular immunopharmaceuticals (SMIP), V_{HH} containing antibodies; and any other polypeptides formed from antibody fragments.

The antigen binding antibodies and fragments encompass single-chain antibody fragments (scFv) that bind to the desired antigen. An scFv may comprise an antibody heavy chain variable region (V_{H}) operably linked to an antibody light chain variable region (V_{L}) wherein the heavy chain variable region and the light chain variable region, together or individually, form a binding site that binds to the antigen. An scFv may comprise a V_{H} region at the amino-terminal end and a V_{L} region at the carboxy-terminal end. Alternatively, scFv may comprise a V_{L} region at the amino-terminal end and a V_{H} region at the carboxy-terminal end. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv). An scFv may optionally further comprise a polypeptide linker between the heavy chain variable region and the light chain variable region.

The antigen binding antibodies and fragments thereof also encompass immunoadhesins. One or more CDRs may be incorporated into a molecule either covalently or noncovalently to make it an immunoadhesin. An immunoadhesin may incorporate the CDR(s) as part of a larger polypeptide chain, may covalently link the CDR(s) to another polypeptide chain, or may incorporate the CDR(s) noncovalently. The CDRs permit the immunoadhesin to specifically bind to the desired antigen.

The antigen binding antibodies and fragments thereof also encompass antibody mimics which may comprise one or more antigen binding portions built on an organic or molecular scaffold (such as a protein or carbohydrate scaffold). Proteins having relatively defined three-dimensional structures, commonly referred to as protein scaffolds, may be used as reagents for the design of antibody mimics. These scaffolds typically contain one or more regions which are amenable to specific or random sequence variation, and such sequence randomization is often carried out to produce libraries of proteins from which desired products may be selected. For example, an antibody mimic can comprise a chimeric non-immunoglobulin binding polypeptide having an immunoglobulin-like domain containing scaffold having two or more solvent exposed loops containing a different CDR from a parent antibody inserted into each of the loops and exhibiting selective binding activity toward a ligand bound by the parent antibody. Non-immunoglobulin protein scaffolds have been proposed for obtaining proteins with novel binding properties.

Antigen specific antibodies or antibody fragments thereof typically bind to the desired antigen with high affinity (e.g., as determined with BIAcore), such as for example with an equilibrium binding dissociation constant (K_{D}) for the antigen of about 15 nM or less, 10 nM or less, about 5 nM or less, about 1 nM or less, about 500 pM or less, about 250 pM or less, about 100 pM or less, about 50 pM or less, or about 25 pM or less, about 10 pM or less, about 5 pM or less, about 3 pM or less about 1 pM or less, about 0.75 pM or less, or about 0.5 pM or less.

Peptides, such as peptide aptamers, can be selected from peptide libraries by screening procedures. In practice, any vector system suitable for expressing short nucleic acid sequences in a eukaryotic cell can be used to express libraries of peptides. In a preferred embodiment, high-titer retroviral packaging systems can be used to produce peptide aptamer libraries. Various vectors, as well as amphotropic and ecotropic packaging cell lines, exist that can be used for production of high titers of retroviruses that infect mouse or human cells. These delivery and expression systems can be readily adapted for efficient infection of any mammalian cell type, and can be used to infect tens of millions of cells in one experiment. Aptamer libraries which may comprise nucleic acid sequences encoding random combinations of a small number of amino acid residues, e.g., 5, 6, 7, 8, 9, 10 or more, but preferably less than 100, more preferably less than 50, and most preferably less than 20, can be expressed in retrovirally infected cells as free entities, or depending on the target of a given screen, as fusions to a heterologous protein, such as a protein that can act as a specific protein scaffold (for promoting, e.g., expressibility, intracellular or intracellular localization, stability, secretability, isolatablitiy, or detectability of the peptide aptamer. Libraries of random peptide aptamers when composed of, for example 7 amino acids, encode molecules large enough to represent significant and specific structural information, and with 10⁷ or more possible combinations is within a range of cell numbers that can be tested.

Preferably, the aptamers are generated using sequence information from the target antigen.

In identifying an aptamer, for example, a population of cells is infected with a gene construct expressing members of an aptamer library, and the ability of aptamers to bind to an antigen is assessed, for instance on a BIAcore platform. Coding sequences of aptamers selected in the first round of screening can be amplified by PCR, re-cloned, and re-introduced into naïve cells. Selection using the same or a different system can then be repeated in order to validate individual aptamers within the original pool. Aptamer coding sequences within cells identified in subsequent rounds of selection can be iteratively amplified and subcloned and the sequences of active aptamers can then be determined by DNA sequencing using standard techniques.

Polypeptides tethered to a synthetic molecular structure are known in the art (Kemp, D. S, and McNamara, P. E., J. Org. Chem., 1985; Timmerman, P. et al., ChemBioChem, 2005). Meloen and co-workers had used tris(bromomethyl)benzene and related molecules for rapid and quantitative cyclisation of multiple peptide loops onto synthetic scaffolds for structural mimicry of protein surfaces (Timmerman, P. et al., ChemBioChem, 2005). Methods for the generation of candidate drug compounds wherein said compounds are generated by linking cysteine containing polypeptides to a molecular scaffold as for example tris(bromomethyl)benzene are disclosed in WO 2004/077062 and WO 2006/078161.

WO2004/077062 discloses a method of selecting a candidate drug compound. In particular, this document discloses various scaffold molecules which may comprise first and second reactive groups, and contacting said scaffold with a further molecule to form at least two linkages between the scaffold and the further molecule in a coupling reaction.

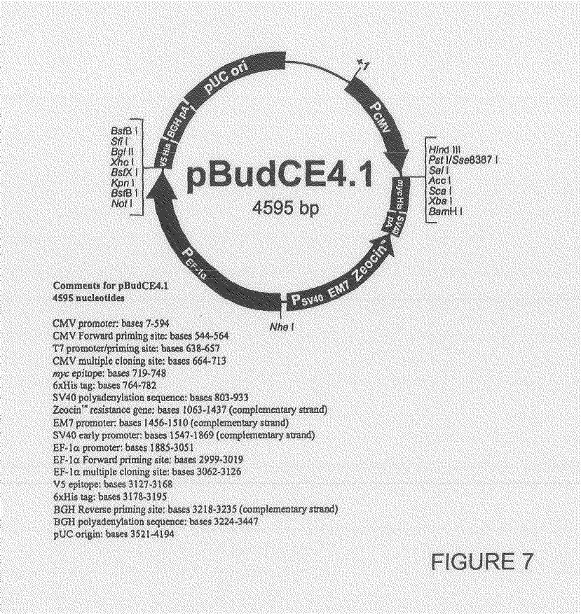

WO2006/078161 discloses binding compounds, immunogenic compounds and peptidomimetics. This document discloses the artificial synthesis of various collections of peptides taken from existing proteins. These peptides are then combined with a constant synthetic peptide having some amino acid changes introduced in order to produce combinatorial libraries. By introducing this diversity via the chemical linkage to separate peptides featuring various amino acid changes, an increased opportunity to find the desired binding activity is provided. FIG. 7 of this document shows a schematic representation of the synthesis of various loop peptide constructs.

International patent application WO2009098450 describes the use of biological selection technology, such as phage display, to select peptides tethered to synthetic molecular structures. In this approach, peptides are expressed on phage, and then reacted under suitable conditions with molecular scaffolds, such that a structurally constrained peptide is displayed on the surface of the phage.

Such structured peptides can be designed to bind to any desired antigen, and can be coupled to a RING domain in order to direct the antigen-ligand complex to the proteasome inside a cell.

Indirect ligands bind to the antigen via a second ligand, which recognises the antigen specifically. For example, the second ligand is an antibody which is specific to the antigen. Ligands described in sections 1a-1c above may be prepared which are specific for immunoglobulins, but which bind thereto in a manner which is not dependent on the binding specificity of the target immunoglobulin. For instance, anti-Fc antibodies, peptides and structured peptides may be prepared. Antibody-binding peptides such as Protein A, Protein G and Protein L can be used.

Tripartite motif (TRIM) proteins constitute a protein family based on a conserved domain architecture (known as RBCC) that is characterized by a RING finger domain, one or two B-box domains, a Coiled-coil domain and a variable C-terminus.

TRIM proteins are implicated in a variety of cellular functions, including differentiation, apoptosis and immunity. A number of TRIM proteins have been found to display antiviral activities or are known to be involved in processes associated with innate immunity. As noted by Carthagena, et al., PLoS One (2009) 4, 3:e4894, TRIM5a is responsible for a species-specific post-entry restriction of diverse retroviruses, including N-MLV and HIV-1, in primate cells, whereas TRIM1/MID2 also displays an anti-retroviral activity which affects specifically N-MLV infection. TRIM22, also known as Staf50, has been shown to inhibit HIV-1 replication, although it is still unclear at what step the block occurs. TRIM28 restricts MLV LTR-driven transcription in murine embryonic cells. Furthermore, the inhibition of a wide range of RNA and DNA viruses by TRIM19/PML has been reported. The most extensive screen performed to date showed that several TRIM proteins, including TRIM11, TRIM31 and TRIM62, can interfere with various stages of MLV or HIV-1 replication. Finally, TRIM25 has been shown to control RIGI-mediated antiviral activity through its E3 ubiquitin ligase activity.

The RING finger of TRIM21, as set forth herein, is responsible for directing bound antibody/antigen complexes to the proteasome. This is due to the E3 ubiquitin ligase activity of the RING domain. Advantageously, therefore, the RING domain used in the present invention has an E3 ligase activity.

The replacement of RING domains with heterologous TRIM domains, exchanging them between IM proteins, is known in the art. See Li et al., J. Virol. (2006) 6198-6206.

RING domains were described by Freemont et al., Cell. 1991 Feb. 8; 64(3):483-4. The domains are believed to function as E3 ligases; see Meroni & Roux, BioEssays 27, 11: 1147-1157 (2005). They are members of the RING-finger (Really Interesting New Gene) domain superfamily, a specialized type of Zn-finger of 40 to 60 residues that binds two atoms of zinc; defined by the ‘cross-brace’ motif C-X2-C-X(9-39)-C-X(1-3)-H-X(2-3)-(N/C/H)-X2-C-X(4-48)C-X2-C. There are two variants within the family, the C3HC4-type and a C3H2C3-type (RING-H2 finger), which have a different cysteine/histidine pattern.

Preferred RING domains are derived from TRIM proteins, and may be part of TRIM proteins. In one embodiment, the present invention provides a TRIM polypeptide in which the B30.2 domain, which imparts its specificity, is replaced with an antigen-specific binding domain. At least the PRYSPRY (B30.2) domain is replaced; other domains may be replaced or omitted, as long as the RING domain E3 Iigase function is conserved.

Instead of, or in addition to, coupling the RING domain of a TRIM polypeptide to the desired antigen, it is possible to stimulate the expression of endogenous TRIM21 within a cell. TRIM21 binds to antibodies with high affinity, and directs the antibody and any bound antigen to the proteasome.

Since TRIM21 binds to the Fc portion of the antibody, if endogenous TRIM21 expression is stimulated by conjugating the ligand to an inducer of TRIM expression, the ligand may comprise a binding site for the PRYSPRY domain of TRIM21. Preferably, it may comprise an antibody Fc region, and in one embodiment it is an antibody. For example, the antibody can be an IgG or IgM antibody.

TRIM21 expression is induced by interferon. In one embodiment, therefore, the inducer of TRIM expression is interferon, or an interferon inducer.

Interferon is preferably type I interferon, for example alpha interferon or beta interferon.

Interferons are known in the art in a number of therapeutic applications, but especially in therapy for HBV and HCV. Interferon derivatives, such as peginterferon (pegylated interferon) and albuferon (interferon conjugated to HSA) are coadministered with antiviral agents, such as nucleoside analogues.

Interferon inducers are known in the art. In general, many vaccine adjuvants act as interferon inducers. These include substances that have been known to act as vaccine adjuvants for many years, including viral antigens, bacterial antigens such as LPS, synthetic polymers usch as poly I:C (e.g. Ampligen®). More recently, it has been shown that agonists of Toll-like receptors (TLRs) are effective inducers of interferon. For example, a number of interferon inducers are known from US2010120799; US2010048520; US2010018134; US2010018132; US2010018131; US2010018130; US2010003280. Moreover, small molecule interferon inducers are being developed, for instance as set forth in Musmuca et al., J. Chem. Inf. Model., 2009, 49 (7), pp 1777-1786.

Methods for attaching a drug or other small molecule pharmaceutical to an antibody fragment are well-known. various peptide conjugation chemistries are established in the art and include bifunctional chemical linkers such as N-succinimidyl (4-iodoacetyl)-aminobenzoate; sulfosuccinimidyl (4-iodoacetyl)-aminobenzoate; 4-succinimidyl oxycarbonyl-[alpha]-(2-pyridyldithio)toluene; sulfosuccinimidyl-6-[[alpha]-methyl-[alpha]-(pyridyldithiol)-toluamido]hexanoate; N-succinimidyl-3-(−2-pyridyldithio)-proprionate; succinimidyl-6-[3(-(-2-pyridyldithio)-proprionamido]hexanoate; sulfosuccinimidyl-6-[3(+2-pyridyldithio)-propionamido]hexanoate; 3-(2-pyridyldithio)-propionyl hydrazide, Ellman's reagent, dichlorotriazinic acid, S-(2-thiopyridyl)-L-cysteine, and the like. Further bifunctional linking molecules are disclosed in U.S. Pat. Nos. 5,349,066; 5,618,528; 4,569,789; 4,952,394; and 5,137,877, as well as Corson et al., ACS Chemical Biology 3, 11, pp 677-692, 2008.

The RING domains and polypeptide ligands, including antibodies, may be conjugated via functional or reactive groups on one (or both) polypeptide(s). These are typically formed from the side chains of particular amino acids found in the polypeptide polymer. Such reactive groups may be a cysteine side chain, a lysine side chain, or an N-terminal amine group or any other suitable reactive group.

Reactive groups are capable of forming covalent bonds to the ligand to be attached. Functional groups are specific groups of atoms within either natural or non-natural amino acids which form the functional groups.

Suitable functional groups of natural amino acids are the thiol group of cysteine, the amino group of lysine, the carboxyl group of aspartate or glutamate, the guanidinium group of arginine, the phenolic group of tyrosine or the hydroxyl group of serine. Non-natural amino acids can provide a wide range of functional groups including an azide, a keto-carbonyl, an alkyne, a vinyl, or an aryl halide group. The amino and carboxyl group of the termini of the polypeptide can also serve as functional groups to form covalent bonds to a desired ligand.

Alternatives to thiol-mediated conjugations can be used to attach a ligand to a polypeptide via covalent interactions. These methods may be used instead of (or in combination with) the thiol mediated methods by producing polypeptides bearing unnatural amino acids with the requisite chemical functional groups, in combination small molecules that bear the complementary functional group, or by incorporating the unnatural amino acids into a chemically or recombinantly synthesised polypeptide when the molecule is being made after the selection/isolation phase.

The unnatural amino acids incorporated into peptides and proteins on phage may include 1) a ketone functional group (as found in para or meta acetyl-phenylalanine) that can be specifically reacted with hydrazines, hydroxylamines and their derivatives (Addition of the keto functional group to the genetic code of Escherichia coli. Wang L, Zhang Z, Brock A, Schultz PG. Proc Natl Acad Sci USA. 2003 Jan. 7; 100(1):56-61; Bioorg Med Chem. Lett. 2006 Oct. 15; 16(20):5356-9. Genetic introduction of a diketone-containing amino acid into proteins. Zeng H, Xie J, Schultz P G), 2) azides (as found in p-azido-phenylalanine) that can be reacted with alkynes via copper catalysed “click chemistry” or strain promoted (3+2) cyloadditions to form the corresponding triazoles (Addition of p-azido-L-phenylalanine to the genetic code of Escherichia coli. Chin J W, Santoro S W, Martin A B, King D S, Wang L, Schultz P G. J Am Chem. Soc. 2002 Aug. 7; 124(31):9026-7; Adding amino acids with novel reactivity to the genetic code of Saccharomyces cerevisiae. Deiters A, Cropp T A, Mukherji M, Chin J W, Anderson J C, Schultz P G. J Am Chem. Soc. 2003 Oct. 1; 125(39):11782-3), or azides that can be reacted with aryl phosphines, via a Staudinger ligation (Selective Staudinger modification of proteins containing p-azidophenylalanine Tsao M L, Tian F, Schultz P G. Chembiochem. 2005 December; 6(12):2147-9), to form the corresponding amides, 4) Alkynes that can be reacted with azides to form the corresponding triazole (In vivo incorporation of an alkyne into proteins in Escherichia coli. Deiters A, Schultz P G. Bioorg Med Chem. Lett. 2005 Mar. 1; 15(5):1521-4), 5) Boronic acids (boronates) than can be specifically reacted with compounds containing more than one appropriately spaced hydroxyl group or undergo palladium mediated coupling with halogenated compounds (Angew Chem Int Ed Engl. 2008; 47(43):8220-3. A genetically encoded boronate-containing amino acid., Brustad E, Bushey M L, Lee J W, Groff D, Liu W, Schultz P G), 6) Metal chelating amino acids, including those bearing bipyridyls, that can specifically co-ordinate a metal ion (Angew Chem Int Ed Engl. 2007; 46(48):9239-42. A genetically encoded bidentate, metal-binding amino acid. Xie J, Liu W, Schultz P G).

Unnatural amino acids may be incorporated into proteins and peptides by transforming E. coli with plasmids or combinations of plasmids bearing: 1) the orthogonal aminoacyl-tRNA synthetase and tRNA that direct the incorporation of the unnatural amino acid in response to a codon, 2) The phage DNA or phagemid plasmid altered to contain the selected codon at the site of unnatural amino acid incorporation (Proc Natl Acad Sci USA. 2008 Nov. 18; 105(46):17688-93, Protein evolution with an expanded genetic code. Liu C C, Mack A V, Tsao M L, Mills J H, Lee H S, Choe H, Farzan M, Schultz P G, Smider V V; A phage display system with unnatural amino acids. Tian F, Tsao M L, Schultz P G, J Am Chem. Soc. 2004 Dec. 15; 126(49):15962-3). The orthogonal aminoacyl-tRNA synthetase and tRNA may be derived from the Methancoccus janaschii tyrosyl pair or a synthetase (Addition of a photocrosslinking amino acid to the genetic code of Escherichia coli. Chin J W, Martin A B, King D S, Wang L, Schultz P G. Proc Natl Acad Sci USA. 2002 Aug. 20; 99(17):11020-4) and tRNA pair that naturally incorporates pyrrolysine (Multistep engineering of pyrrolysyl-tRNA synthetase to genetically encode N(epsilon)-(o-azidobenzyloxycarbonyl)lysine for site-specific protein modification. Yanagisawa T, Ishii R, Fukunaga R, Kobayashi T, Sakamoto K, Yokoyama S. Chem. Biol. 2008 Nov. 24; 15(11):1187-97; Genetically encoding N(epsilon)-acetyllysine in recombinant proteins. Neumann H, Peak-Chew S Y, Chin J W. Nat Chem. Biol. 2008 April; 4(4):232-4. Epub 2008 Feb. 17). The codon for incorporation may be the amber codon (UAG) another stop codon (UGA, or UAA), alternatively it may be a four base codon. The aminoacyl-tRNA synthetase and tRNA may be produced from existing vectors, including the pBK series of vectors, pSUP (Efficient incorporation of unnatural amino acids into proteins in Escherichia coli. Ryu Y, Schultz P G. Nat. Methods. 2006 April; 3(4):263-5) vectors and pDULE vectors (Nat. Methods. 2005 May; 2(5):377-84. Photo-cross-linking interacting proteins with a genetically encoded benzophenone. Farrell I S, Toroney R, Hazen J L, Mehl R A, Chin J W). The E. coli strain used will express the F′ pilus (generally via a tra operon). When amber suppression is used the E. coli strain will not itself contain an active amber suppressor tRNA gene. The amino acid will be added to the growth media, preferably at a final concentration of 1 mM or greater. Efficiency of amino acid incorporation may be enhanced by using an expression construct with an orthogonal ribosome binding site and translating the gene with ribo-X(Evolved orthogonal ribosomes enhance the efficiency of synthetic genetic code expansion. Wang K, Neumann H, Peak-Chew S Y, Chin J W. Nat. Biotechnol. 2007 July; 25(7):770-7). This may allow efficient multi-site incorporation of the unnatural amino acid providing multiple sites of attachment to the ligand.

Such methods are useful to attach RING domains to antibodies and other ligands, including non-peptide ligands. They are also useful for attaching small molecule interferon inducers, and other inducers of TRIM21 expression.

Techniques for conjugating antibodies to drugs and other compounds are also described in Carter & Senter, Cancer Journal: May/June 2008—Volume 14—Issue 3—pp 154-169; Ducry and Stump, Bioconjugate Chem., 2010, 21 (1), pp 5-13.

Alternatively, bispecific antibodies may be used. For example, bispecific domain antibodies are known in the art, and are useful for targeting both a desired antigen and a RING domain, or a polypeptide which may comprise a RING domain.

The half-life of antibody conjugates in the serum is dependent no a number of factors, but smaller antibody fragments tend to be eliminated quickly from the circulation. Accordingly, smaller constructs, for example which may comprise a domain antibody and a RING domain, are advantageously coupled to a polypeptide which increases serum half-life. For example, they can be coupled to HSA. Preferably, the bond to HSA is labile, for example having a defined half life, such that the construct is released from the HSA when bound to a cell, and is internalised without the HSA. A useful approach is to use a multispecific ligand construct, such that the ligand also binds HSA, maintaining it in circulation. The affinity of the ligand for HSA can be tailored such that the ligand can be internalised by the cell as appropriate.

Therapeutic antibodies are well known in the art. TRIM21 binds to the Fc portion of IgG and IgM antibodies, and coadministration thereof to a subject is effective in promoting the destruction of pathogens by cells.

Table 1 sets forth existing antibody drugs which are available for the treatment of pathogenic infections. Coadministration of TRIM21 is indicated for treatment with such drugs.

The polypeptide coadministered with the antibody drug preferably may comprise a TRIM21 PRYSPRY domain and a RING domain, capable of acting as an E3 ligase. However, other immunoglobulin-specific ligands can be used, such as protein A, protein G or protein L, or anti-Fc peptides, which bind to immunoglobulins in a manner independent of the antibody's target specificity.

Preferably, the polypeptide also may comprise a coiled coil domain and/or a B-box domain. In a preferred embodiment, it is a substantially complete TRIM21 polypeptide.

TRIM21 is preferably human TRIM21, as set forth in SEQ ID No. 1; See Tanaka, M., et al., Histochem. Cell Biol. 133 (3), 273-284 (2010).

The invention encompasses modified derivatives of TIM21, which conserve at least the antibody-binding and E3 ligase functions. For example, the invention encompasses substitutions, additions or deletions within the amino acid sequence of TRIM21, as long as the required functions are sufficiently maintained. Polypeptides may share at least 50%, 55%, 60%, 65%, 70%, 75%, 80%, 85%, 90%, 95%, 96%, 97%, 98% or 99% sequence identity (homology) with SEQ ID NO. 1.

Mutation on the polypeptides of the invention can be targeted to certain domains thereof. Higher levels of conservation of sequence identity are required, for instance, in the PRYSPRY domain. This domain is responsible for antibody binding by the polypeptide. Lower levels of identity are generally required, for example, in the RING domain. RING domains are widespread in the genome, and have a conserved E3 ligase function. Advantageously, the consensus sequence, C-X2-C-X(9-39)-C-X(1-3)-H-X(2-3)-(N/C/H)-X2-C-X(4-48)C-X2-C, is maintained.

TABLE 1
antibody anti-infective drugs

| Drug | Originator | Therapy Areas |
|---|---|---|
| anthrax monoclonal | Aprogen Inc | Bacillus anthracis infection |
| antibodies, Aprogen | | |
| immune globulin (anthrax), | Cangene Corp | Bacillus anthracis infection |
| Cangene | | |
| MDX-1303 | Medarex Inc | Bacillus anthracis infection |
| raxibacumab | Cambridge Antibody | Bacillus anthracis infection |
| | Technology Group plc | |
| AVP-21D9 | AVANIR Pharmaceuticals Inc | Bacillus anthracis infection |
| anthrax immune globulin | Emergent BioSolutions Inc | Bacillus anthracis infection |
| therapy (intravenous), | | |
| Emergent | | |
| ETI-204 | Elusys Therapeutics Inc | Bacillus anthracis infection |
| camel-derived anti-anthrax | Canopus BioPharma Inc | Bacillus anthracis infection |
| toxin antibodies, Canopus | | |
| BioPharma | | |
| human monoclonal antibodies | IQ Therapeutics BV | Bacillus anthracis infection |
| (anthrax), IQ | | |
| antitoxin antibodies, Alexion | Alexion Pharmaceuticals Inc | Bacillus anthracis infection; |
| | | Clostridium botulinum infection |
| STI-001 program (mAbs | Sorrento Therapeutics Inc | Bacterial infection; MRSA infection |
| targeting S aureus auto- | | |
| inducing peptides), Sorrento | | |
| immune globulin (botulism), | Cangene Corp | Clostridium botulinum infection |
| Cangene | | |
| botulinum antibody therapy, | Emergent BioSolutions Inc | Clostridium botulinum infection |
| Emergent/HPA | | |
| botulinum toxin antibodies, | Humanyx Ltd | Clostridium botulinum infection |
| Humanyx | | |
| XOMA-3AB | XOMA Ltd | Clostridium botulinum infection |
| MDX-066 | Medarex Inc | Clostridium difficile infection; |
| | | Diarrhea |
| MK-3415A | Medarex Inc | Clostridium difficile infection; |
| | | Diarrhea |
| C difficile toxin A/B mAbs | Progenics Pharmaceuticals Inc | Clostridium difficile infection |
| (infection), Progenics | | |
| anti-Clostridium difficile toxin | EPIcyte Pharmaceutical Inc | Clostridium difficile infection; |
| antibody, EPIcyte | | Diarrhea |
| Diffistat-G | Let's Talk Recovery Inc | Clostridium difficile infection; |
| | | Diarrhea |
| ShigamAbs | Sunol Molecular Corp | Bacterial infection; |
| | | Escherichia coli infection; |
| | | Hemolytic uremic syndrome |
| anti-Ebola virus recombinant | State Research Center of | Ebola virus infection |
| IgG monoclonal antibodies, | Virology and Biotechnology | |
| SRC VB Vector | VECTOR | |
| edobacomab | XOMA Ltd | Endotoxic shock; Gram |
| | | negative bacterium infection; |
| | | Sepsis; Septic shock |
| anti-HBV therapy (RAS), | ChronTech Pharma AB | Hepatitis B virus infection |
| Tripep | | |
| GC-1102 | Green Cross Corp | Hepatitis B virus infection |
| Nabi-HB (intramuscular) | Nabi Biologics | Hepatitis B virus infection |
| Nabi-HB (intravenous) | Nabi Biologics | Hepatitis B virus infection |
| HepeX-B | XTL Biopharmaceuticals Ltd | Hepatitis B virus infection |
| HuMax-HepC | Genmab A/S | Hepatitis C virus infection |
| MBL-HCV1 | Massachusetts Biologic | Hepatitis C virus infection |
| | Laboratories | |
| XTL-6865 | XTL Biopharmaceuticals Ltd | Hepatitis C virus infection |
| anti-HCV antibodies, XTL | XTL Biopharmaceuticals Ltd | Hepatitis C virus infection |
| GNI-104 | GENimmune NV | Hepatitis C virus infection |
| CDX-2401 | Celldex Therapeutics Inc | HIV infection |
| | (pre-merger) | |
| monoclonal antibody dB4 | United Biomedical Inc | HIV infection |
| (HIV infection), United | | |
| Biomedical | | |
| human monoclonal antibodies | Theraclone Sciences Inc | HIV infection |
| (HIV infection), Theraclone | | |
| Sciences | | |
| AD-439 | Tanox Inc | HIV infection; Viral infection |
| PRO-367 | Progenics Pharmaceuticals Inc | HIV infection |
| anti-HIV catalytic antibody, | Hesed Blamed Inc | HIV infection |
| Hesed Biomed | | |
| anti-gp120-trigger MAb, | Medarex Inc | HIV infection |
| Medarex | | |
| HIV monoclonals, RepliGen | Repligen Corp | HIV infection |
| microbicide monoclonal | Polymun Scientific | HIV infection |
| antibody triple combination | Immunobiologische | |
| (vaginal gel formulation, HIV | Forschung GmbH | |
| infection), Polymun | | |
| monoclonal antibody triple | Polymun Scientific | HIV infection |
| combination (intravenous, | Immunobiologische | |
| HIV infection), Polymun | Forschung GmbH | |
| PRO-542 | Progenics Pharmaceuticals Inc | HIV infection |
| MDX-240 | Medarex Inc | HIV infection |
| scFv-F240, Beth Israel/ | Beth Israel Deaconess | HIV-1 infection |
| Harvard Medical School | Medical Center | |
| gp41-based HIV therapeutic | Bioclonetics Inc | HIV infection |
| antibodies, BTG | | |
| D5 monoclonal antibody, | Cambridge Antibody | HIV infection |
| Cambridge Antibody | Technology Group plc | |
| Technology/Merck & Co | | |
| gene therapy (HIV), Intracel | Intracel Corp | HIV infection |
| anti-p17 Fab intrabodies (HIV | Dana-Farber Cancer Institute | HIV infection |
| infection), Dana-Farber | Inc | |
| monoclonal antibody (HIV), | Eukarion Inc | HIV infection |
| Eukarion | | |
| anti-TAT monoclonal | BioInvent International AB | HIV infection |
| antibodies (HIV infection), | | |
| BioInvent | | |
| HX-8 | EPIcyte Pharmaceutical Inc | Herpes simplex virus infection |
| mapp-66 | Mapp Biopharmaceutical Inc | HIV infection; Herpes |
| | | simplex virus infection |
| H5 subtype avian influenza | Xiamen Innovax Biotech Co | Influenza virus infection |
| vaccine (monoclonal | Ltd | |
| antibody), Xiamen Innovax | | |
| Biotech | | |
| H5N1 and H1N1 influenza | Crucell NV | Influenza virus infection |
| monoclonal antibody therapy | | |
| (PER.C6), Crucell | | |
| anti-M2 antibodies | Kirin Pharma USA Inc | Influenza virus infection |
| (influenza), Kyowa Hakko | | |
| Kirin | | |
| anti-M2e human monoclonal | Theraclone Sciences Inc | Influenza virus infection |
| antibody (seasonal/pandemic | | |
| influenza), Theraclone | | |
| Sciences/Zenyaku Kogyo | | |
| pagibaximab | Biosynexus Inc | Enterococcus infection; |
| | | Staphylococcus infection |
| human recombinant anti- | AVANIR Pharmaceuticals Inc | Staphylococcus infection |
| lipoteichoic acid monoclonal | | |
| antibodies (Staphylococcus | | |
| infection), Avanir | | |
| Mab-338 | MedImmune LLC | Metapneumovirus infection |
| anti-phosphatidylserine | Peregrine Pharmaceuticals Inc | Viral infection |
| antibody (viral infection), | | |
| Peregrine | | |
| AT-005, Affitech | Affitech A/S | Viral infection |
| IgNar antibody (malaria), | AdAlta Pty Ltd | Plasmodium infection |
| AdAlta | | |
| F-598 (serious bacterial | Harvard Medical School | Bacterial infection |
| infection), Alopexx | | |
| Pharmaceuticals | | |
| monoclonal antibodies | Baxter International Inc | Pseudomonas aeruginosa infection |
| (pseudomonas), | | |
| Baxter/PathoGenesis | | |
| KBPA-102 | Kenta Biotech Ltd | Pseudomonas aeruginosa infection |
| panobacumab | Berna Biotech AG | Pseudomonas aeruginosa infection |
| KBPA-103 | Kenta Biotech Ltd | Pseudomonas aeruginosa infection |
| KBPA-104 | Kenta Biotech Ltd | Pseudomondales infection |
| monoclonal antibody (inhaler, | Harvard Medical School | Pseudomonas aeruginosa infection |
| Pseudomonas lung infection), | | |
| Aridis | | |
| PA-04 | EPIcyte Pharmaceutical Inc | Lung infection; Pseudomonas |
| | | aeruginosa infection |
| KB-001 | University of California San | Pseudomonas infection |
| | Francisco | |
| palivizumab | MedImmune LLC | Respiratory syncytial virus |
| | | infection |
| motavizumabYTE | MedImmune LLC | Respiratory syncytial virus |
| | | infection |
| MEDI-557 | MedImmune LLC | Respiratory syncytial virus |
| | | infection |
| anti-RSV human MAbs, IDEC | IDEC Pharmaceuticals Corp | Respiratory syncytial virus |
| | | infection |
| felvizumab | Scotgen Biopharmaceuticals | Respiratory syncytial virus |
| | Inc | infection |
| EPI-19 | EPIcyte Pharmaceutical Inc | Respiratory syncytial virus |
| | | infection |
| SYM-003 | Symphogen A/S | Respiratory syncytial virus |
| | | infection |
| CR-3014 | Crucell NV | SARS coronavirus infection |
| SARS coronavirus spike | National Institutes of Health | SARS coronavirus infection |
| glycoprotein antibodies | | |
| (SARS), National Institutes of | | |
| Health | | |
| urtoxazumab | Teijin Ltd | Escherichia coli infection; |
| | | Hemolytic uremic syndrome; |
| | | Hemorrhagic enteritis |
| tefibazumab | Inhibitex Inc | Staphylococcus aureus infection |
| anti-S aureus humanized | Intercell AG | Staphylococcus aureus infection |
| monoclonal antibodies, Merck | | |
| & Co | | |
| anti-Staphylococcus | Integrated BioTherapeutics | Staphylococcus infection |
| enterotoxin B hyperimmune | Inc | |
| globulin, Integrated | | |
| BioTherapeutics | | |
| ETI-211 | Elusys Therapeutics Inc | Staphylococcus aureus infection |
| superantigen toxin therapy | Synergy Pharmaceuticals Inc | Sepsis; Shock; |
| (antibodies), Callisto | | Staphylococceae infection; |
| | | Streptococcaceae infection; |
| | | Toxicity |
| Tetagam | CSL Behring | Clostridium tetani infection |
| KBAB-401 | Kenta Biotech Ltd | Acinetobacter infection |
| anti-anthrax antibodies, | Verenium Corp | Bacillus anthracis infection |
| Verenium | | |
| polyclonal anti-RcPA IgG, | NEXTherapeutics Inc | Bacillus anthracis infection |
| NEXTherapeutics | | |
| ACE-710 | ACE BioSciences A/S | Aspergillus fumigatus infection |
| SYM-006 | Symphogen A/S | Bacterial infection |
| immune globulin | Cangene Corp | Burkholderia infection |
| (Burkholderia, monoclonal), | | |
| Cangene | | |
| CAL-401 | Calmune Corp | Candida albicans infection |
| XTL-Cand-MAb | XTL Biopharmaceuticals Ltd | Candida infection |
| Candistat-G | Let's Talk Recovery Inc | Candida albicans infection |
| monoclonal antibodies (C | Inhibitex Inc | Candida albicans infection |
| albicans), Inhibitex | | |
| OPHD-001 | Ophidian Pharmaceuticals Inc | Clostridium difficile infection |
| immunotoxin (INAx, CMV | Inagen Aps | Cytomegalovirus infection |
| infection), Inagen | | |
| sevirumab | Novartis AG | Bone marrow transplantation; |
| | | CMV retinitis; |
| | | Cytomegalovirus infection |
| human monoclonal antibody | Humabs LLC | Cytomegalovirus infection |
| (CMV infection), Humabs | | |
| human monoclonal antibodies | Theraclone Sciences Inc | Cytomegalovirus infection |
| (CMV infection in organ | | |
| transplantation), Theraclone | | |
| Sciences | | |
| CytoGam | MedImmune LLC | Cytomegalovirus infection |
| truly human monoclonal | RiboVax Biotechnologies SA | Cytomegalovirus infection |
| antibodies (CMV infection), | | |
| RiboVax Biotechnologies | | |
| human monoclonal antibodies | Evec Inc | Cytomegalovirus infection |
| (cytomegalovirus), Evec | | |
| regavirumab | Teijin Ltd | Cytomegalovirus infection; |
| | | Immune disorder |
| monoclonal antibody (CMV), | Scotgen Biopharmaceuticals | Cytomegalovirus infection |
| Scotgen | Inc | |
| SDZ-89-104 | Novartis AG | Bone marrow transplantation; |
| | | Cytomegalovirus infection |
| immunoglobulin (CMV), | Cangene Corp | Cytomegalovirus infection |
| Cangene | | |
| CryptoGAM | ImmuCell Corp | Cryptosporidium infection |
| bovine immunoglobulin | Let's Talk Recovery Inc | Diarrhea; Parasitic infection |
| concentrate-C parvum, | | |
| GalaGen | | |
| Sporidin-G | Let's Talk Recovery Inc | Cryptosporidium infection; |
| | | Diarrhea |
| therapeutic antibody program | Sentinext Therapeutics Sdn | Dengue virus infection |
| (dengue virus infection), | Bhd | |
| Sentinext | | |
| monoclonal antibody (dengue | MacroGenics Inc | Dengue virus infection |
| virus), MacroGenics | | |
| Enterostat-G | Alcon Inc | Diarrhea; Escherichia coli |
| | | infection |
| BWPT-302 | Biomune Systems Inc | Escherichia coli infection |
| TravelGAM | ImmuCell Corp | Escherichia coli infection |
| E coli antibody, Mutabilis | Mutabilis SA | Escherichia coli infection |
| anti-Ebola virus monoclonal | Mapp Biopharmaceutical Inc | Ebola virus infection |
| antibodies (Ebola virus | | |
| infection), MAPP | | |
| immune globulin | Cangene Corp | Filovirus infection |
| (Ebola/Marburg, polyclonal), | | |
| Cangene | | |
| immune globulin | Cangene Corp | Filovirus infection |
| (Ebola/Marburg, monoclonal), | | |
| Cangene | | |
| Enterococcus MAb, Inhibitex | Inhibitex Inc | Enterobacteriaceae infection |
| F10 (neutralizing antibody, | Harvard Medical School | Influenza virus infection |
| group 1 influenza A | | |
| infection), Harvard Medical | | |
| School/Dana-Farber Cancer | | |
| Institute/XOMA/SRI | | |
| International | | |
| anti-hantavirus monoclonal | Huazhong University of | Hantavirus infection |
| antibody, Huazhong | Science and Technology | |
| University | | |
| HAV human antibody, | Aprogen Inc | Hepatitis A virus infection |
| Aprogen | | |
| Beriglobin | Aventis Behring LLC | Hepatitis A virus infection |
| HepaGam B | Cangene Corp | Hepatitis B virus infection |
| tuvirumab | Novartis AG | Hepatitis B virus infection; |
| | | Transplant rejection |
| Omri-Hep-B | OMRIX Biopharmaceuticals | Hepatitis B virus infection |
| | SA | |
| HBV humanized antibodies, | Aprogen Inc | Hepatitis B virus infection |
| Aprogen | | |
| anti-HBV antibody, AltaRex | AltaRex Medical Corp | Hepatitis B virus infection |
| Hepatitis B Hyperimmune | Kedrion SpA | Hepatitis B virus infection |
| C-Immune | Virionics Corp | Hepatitis C virus infection |
| fully-human monoclonal | Stanford University | Hepatitis C virus infection |
| antibodies (hepatitis C virus), | | |
| Crucell | | |
| human monoclonal antibodies | Theraclone Sciences Inc | Hepatitis C virus infection |
| (HCV infection), Theraclone | | |
| Sciences | | |
| Civacir | Nabi Biopharmaceuticals | Hepatitis C virus infection |
| truly human monoclonal | RiboVax Biotechnologies SA | Hepatitis C virus infection |
| antibodies (HCV infection), | | |
| RiboVax Biotechnologies | | |
| anti-HCV hyperimmune, | Cangene Corp | Hepatitis C virus infection |
| Cangene | | |
| Pylorimune-G | Chiron Vaccines Co | Gastrointestinal ulcer; |
| | | Helicobacter pylori infection |
| bovine antibody-based | Erasmus Universiteit | Helicobacter pylori infection |
| immunotherapy (Helicobacter | Rotterdam | |
| pylori infection), Erasmus | | |
| Universiteit Rotterdam | | |
| KD-247 | Chemo-Sero-Therapeutic | HIV infection |
| | Research Inst | |
| MEDI-488 | MedImmune LLC | HIV infection |
| F-105 | Centocor Ortho Biotech Inc | HIV infection |
| TG-102 | TargetGen Biotechnology | HIV infection |
| sCD4-17b | National Institute of Allergy | HIV infection |
| | and Infectious Diseases | |
| PassHIV, Verigen | Verigen Inc | HIV infection |
| HIV-IG | Nabi Biopharmaceuticals | HIV infection |
| G3.519-PAP-S | Tanox Inc | HIV infection |
| hNM01 | Nissin Foods Holdings Co Ltd | HIV infection |
| R7V therapeutic antibody | URRMA Biopharma Inc | HIV infection |
| program, URRMA | | |
| monoclonal antibodies (HIV), | Scotgen Biopharmaceuticals | HIV infection |
| SRD Pharmaceuticals | Inc | |
| HIV-NeutraGAM | Nabi Biopharmaceuticals | HIV infection |
| human monoclonal antibody | Humabs LLC | HIV infection |
| (HIV infection), Humabs | | |
| HIV monoclonals, Roche | Roche Holding AG | HIV-1 infection |
| HIV neutralizing antibodies | Biotherapix SLU | HIV infection |
| (HIV infection), Biotherapix | | |
| anti-HIV therapy, EPIcyte | EPIcyte Pharmaceutical Inc | HIV infection |
| A-221-Immune cHIV | Stratus Research Labs Inc | HIV infection |
| HIV antibodies, Unviersity of | University of Southern | HIV-1 infection |
| Southern California | California | |
| HIV immune globulin, | Chiron Corp | HIV infection |
| Chiron. | | |
| STAR fusion agents (viral | Massachusetts General | Cytomegalovirus infection; |
| infections), Altor | Hospital | HIV infection; Hepatitis C |
| | | virus infection; Infection |
| rhinovirus therapy, EPIcyte | EPIcyte Pharmaceutical Inc | Rhinovirus infection |
| monoclonals, Cambridge | Cambridge Biotech Corp | Cytomegalovirus infection; |
| Biotech | | Herpes simplex virus |
| | | infection; Viral infection |
| AC-8 | Calmune Corp | Herpetic keratitis |
| CAL-102-R | Calmune Corp | Herpes simplex virus |
| | | infection; Ocular disease |
| CAL-103-R | Calmune Corp | Herpes simplex virus |
| | | infection; Ocular disease |
| SMART anti-HSV MAb, PDL | Novartis AG | Herpes simplex virus infection |
| monoclonal antibody therapy | Celltrion Inc | Influenza virus infection |
| (H1N1/H5N1 influenza), | | |
| Celltrion | | |
| influenza therapy, | NanoViricides Inc | Influenza virus infection |
| NanoViricides | | |
| H5N1 influenza mAb therapy, | MacroGenics Inc | Influenza virus infection |
| MacroGenics | | |
| human monoclonal antibody | Humabs LLC | Influenza virus infection |
| (H5N1 infection), Humabs | | |
| camel-derived anti-influenza | Canopus BioPharma Inc | Influenza virus infection |
| virus antibodies, Canopus | | |
| ABC-120 | AERES Biomedical Ltd | Plasmodium infection |
| CAL-201 | Calmune Corp | Metapneumovirus infection |
| metapneumovirus antibody, | ViroNovative BV | Viral respiratory tract infection |
| MedImmune | | |
| monoclonal antibodies | Affitech A/S | Neisseria meningitidis infection |
| (meningitis), Affitech | | |
| nebacumab | Centocor Ortho Biotech Inc | Neisseria meningitidis infection; |
| | | Sepsis |
| henipavirus-neutralizing | National Cancer Institute | Henipavirus infection |
| antibody, NCI | | |
| immunotoxins (INAx, | Inagen Aps | Parasitic infection |
| parasitic infections), Inagen | | |
| CAL-202 | Calmune Corp | Parainfluenza virus infection |
| PCP-Scan | Immunomedics Inc | Fungal pneumonia; Fungal |
| | | respiratory tract infection; |
| | | Pneumocystis carinii infection |
| antibacterial antibodies, | InterMune Inc | Pseudomonas aeruginosa |
| InterMune | | infection; Staphylococcus |
| | | aureus infection |
| polyclonal IgY antibody (oral, | Immunsystem IMS AB | Pseudomonas aeruginosa infection |
| Pseudomonas aeruginosa), | | |
| Immunsystem | | |
| XTL-Pseudomonas-MAb | XTL Biopharmaceuticals Ltd | Pseudomonas aeruginosa infection |
| monoclonal antibody (P | Scotgen Biopharmaceuticals | Pseudomonas aeruginosa infection |
| aeruginosa), Scotgen | Inc | |
| Pseudomonas aeruginosa | Cytovax Biotechnologies Inc | Pseudomonas aeruginosa infection |
| MAb, Millenium | | |
| HyperGAM, Nabi | Nabi Biopharmaceuticals | Pseudomonas aeruginosa infection |
| MS-705 | Mitsui Pharmaceuticals Inc | Pseudomonas aeruginosa infection |
| monoclonal antibody vaccine | Massachusetts Biologic | Rabies virus infection |
| (rabies), MBL/Serum Institute | Laboratories | |
| of India | | |
| foravirumab + rafivirumab | Crucell NV | Rabies virus infection |
| human anti-rabies mAb, | Molecular Targeting | Rabies virus infection |
| Molecular Targeting | Technologies Inc | |
| Technology/North China | | |
| Pharmaceutical Group Corp | | |
| Berirab | CSL Behring | Rabies virus infection |
| monoclonal antibody (rabies), | Scotgen Biopharmaceuticals | Rabies virus infection |
| Scotgen | Inc | |
| Rotastat-G | Alcon Inc | Diarrhea; Viral infection |
| RespiGam | MedImmune LLC | Respiratory syncytial virus |
| | | infection |
| ALX-0171 | Ablynx NV | Respiratory syncytial virus |
| | | infection |
| anti-RSV mAb, | Trellis Bioscience Inc | Respiratory syncytial virus |
| Trellis/MedImmune | | infection |
| HumaRESP | Intracel Corp | Respiratory syncytial virus |
| | | infection |
| RSV human antibodies, | Aprogen Inc | Respiratory syncytial virus |
| Aprogen | | infection |
| KBRV-201 | Kenta Biotech Ltd | Respiratory syncytial virus |
| | | infection; Viral respiratory |
| | | tract infection |
| CAL-203 | Calmune Corp | Respiratory syncytial virus |
| | | infection |
| HNK-20 | Acambis Inc | Respiratory syncytial virus |
| | | infection |
| EGX-220 | EvoGenix Pty Ltd | Respiratory syncytial virus |
| | | infection |
| human monoclonal antibody | Humabs LLC | SARS coronavirus infection |
| (SARS infection), Humabs | | |
| SARS coronavirus antibody, | Medarex Inc | SARS coronavirus infection |
| Medarex/University of | | |
| Massachusetts | | |
| anti-SARS antibodies, | Verenium Corp | SARS coronavirus infection |
| Verenium | | |
| immune globulin (SARS), | Cangene Corp | SARS coronavirus infection |
| Cangene | | |
| hyperimmune globulins | Advantek Biologies Ltd | SARS coronavirus infection |
| (SARS), Advantek | | |
| ACY-111 | Acceptys Inc | Vaccinia virus infection; |
| | | Variola virus infection |
| Veronate | BioResearch Ireland | Bacterial infection; Candida |
| | | infection; Staphylococcus |
| | | aureus infection; |
| | | Staphylococcus infection |
| human monoclonal antibodies | Crucell NV | Bacterial infection |
| (Enterococcus/Staphylococcus | | |
| infection), | | |
| Crucell/MedImmune | | |
| KBSA-301 | Kenta Biotech Ltd | Staphylococcus aureus infection |
| KBSA-302 | Kenta Biotech Ltd | Staphylococcus aureus infection |
| Saurestat | Strox Biopharmaceuticals | Staphylococcus aureus infection |
| | LLC | |
| SX5 | Strox Biopharmaceuticals | Staphylococcus aureus infection |
| | LLC | |
| SX8 | Strox Biopharmaceuticals | Staphylococcus aureus infection |
| | LLC | |
| anti-staphylococcus | MedImmune LLC | Bacterial infection |
| heteropolymer (HP) | | |
| monoclonal antibody | | |
| (infection), MedImmune | | |
| Staphguard | Excelimmune Inc | Staphylococcus aureus infection |
| Aurograb | NeuTec Pharma plc | Staphylococcus aureus infection |
| SA-IGIV | Inhibitex Inc | Staphylococcus aureus infection |
| SE-MAb, Inhibitex/ | Inhibitex Inc | Staphylococcus infection |
| BioResearch | | |
| antibacterial antibodies, | Haptogen Ltd | Bacterial infection; MRSA infection |
| Wyeth Pharmaceuticals/ | | |
| DaeWoong | | |
| XTL-Staph-MAb | XTL Biopharmaceuticals Ltd | Staphylococcus aureus infection |
| anti-S agalactiae humanized | Intercell AG | Streptococcus agalactiae infection |
| monoclonal antibodies, | | |
| Intercell | | |
| hemolytic disease therapy, | Zenyth Therapeutics Ltd | Hemolytic anemia; Hemolytic |
| AMRAD | | streptococcus infection |
| antibody therapy | ACE Bio Sciences A/S | Streptococcus pneumoniae infection |
| (Streptococcus pneumoniae | | |
| infection), ACE | | |
| Biosciences/Crucell | | |
| anti-S pneumoniae humanized | Intercell AG | Streptococcus pneumoniae infection |
| monoclonal antibodies, | | |
| Intercell/Kyowa Hakko Kirin | | |
| CaroRx | Planet Biotechnology Inc | Dental caries; Streptococcus mutans |
| | | infection |
| Streptococcus mutans therapy, | EPIcyte Pharmaceutical Inc | Dental caries; Streptococcus mutans |
| EPIcyte | | infection |
| Streptococcus pneumoniae | Cytovax Biotechnologies Inc | Streptococcus pneumoniae infection |
| mAb, Cytovax | | |
| anti-S pyogenes humanized | Intercell AG | Group A Streptococcus infection |
| monoclonal antibodies, Merck | | |
| & Co | | |
| anti-tick-borne encephalitis | State Research Center of | Flavivirus infection |
| virus monoclonal antibodies, | Virology and Biotechnology | |
| SRC VB Vector | VECTOR | |
| anti-smallpox monoclonal | Kyowa Hakko Kirin Co Ltd | Variola virus infection |
| antibodies, Kyowa Hakko | | |
| Kirin | | |
| SYM-002 | Symphogen A/S | Vaccinia virus infection |
| human monoclonal antibody | US Army Medical Research | Variola virus infection |
| (smallpox), USAMRIID/ | Institute of Infectious Diseases | |
| BioFactura | | |
| monoclonal antibody cocktail | MacroGenics Inc | Variola virus infection |
| (smallpox), MacroGenics | | |
| VariZIG | Cangene Corp | Neuralgia; Varicella zoster |
| | | virus infection |
| monoclonal antibodies | Affitech A/S | Varicella zoster virus infection |
| (Varicella zoster), Affitech | | |
| monoclonal antibody | Scotgen Biopharmaceuticals | Varicella zoster virus infection |
| (varicella), Scotgen | Inc | |
| varicella zoster virus MAbs, | Teijin Ltd | Varicella zoster virus infection |
| Teijin | | |
| anti-VZV MAb (humanized), | Novartis AG | Varicella zoster virus infection |
| PDL | | |
| Varicellon | CSL Behring | Varicella zoster virus infection |
| Western equine encephalitis | ViRexx Medical Corp | Western equine encephalitis |
| virus vaccine (antibody, | | virus infection |
| Chimigen), Paladin | | |
| WNV-HP | Elusys Therapeutics Inc | Viral infection; West Nile |
| | | virus infection |
| Omr-IgG-am | OMRIX Biopharmaceuticals | Immune deficiency; West Nile |
| | SA | virus infection |
| immune globulin (West Nile | Cangene Corp | West Nile virus infection |
| virus), Cangene | | |
| CR-4374 | Crucell NV | West Nile virus infection |
| anti-plague antibodies, | Verenium Corp | Yersinia pestis infection |
| Verenium | | |
| MGAWN-1 | Washington University in St | West Nile virus infection |
| | Louis | |

Generally, the compounds according to the invention will be utilised in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The compounds of the present invention may be used as separately administered compositions or in conjunction with other agents. These can include further antibodies, antibody fragments and conjugates, and various immunotherapeutic drugs, such as cylcosporine, methotrexate, adriamycin or cisplatinum, and immunotoxins. Pharmaceutical compositions can include “cocktails” of various cytotoxic or other agents in conjunction with the selected antibodies, receptors or binding proteins thereof of the present invention, or even combinations of selected polypeptides according to the present invention having different specificities, such as polypeptides selected using different target ligands, whether or not they are pooled prior to administration.

The route of administration of pharmaceutical compositions according to the invention may be any of those commonly known to those of ordinary skill in the art. For therapy, including without limitation immunotherapy, the selected antibodies, receptors or binding proteins thereof of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. The dosage and frequency of administration will depend on the age, sex and condition of the patient, concurrent administration of other drugs, counterindications and other parameters to be taken into account by the clinician.

The compounds of this invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that use levels may have to be adjusted upward to compensate.

The compositions containing the present peptide ligands or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of selected peptide ligand per kilogram of body weight, with doses of 0.05 to 2.0 mg/kg/dose being more commonly used. For prophylactic applications, compositions containing the present peptide ligands or cocktails thereof may also be administered in similar or slightly lower dosages.

A composition containing a compound according to the present invention may be utilised in prophylactic and therapeutic settings to aid in the alteration, inactivation, killing or removal of a select target cell population in a mammal. In addition, the selected repertoires of polypeptides described herein may be used extracorporeally or in vitro selectively to kill, deplete or otherwise effectively remove a target cell population from a heterogeneous collection of cells. Blood from a mammal may be combined extracorporeally with the selected peptide ligands whereby the undesired cells are killed or otherwise removed from the blood for return to the mammal in accordance with standard techniques.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Materials & Methods

### Cells Lines

HEK293T, HeLa, TE671, QT35 and HT1080 were maintained in Dulbecco's modification of Eagle's medium (DMEM) supplemented with 10% fetal bovine serum and 100 IU/ml penicillin and 100 μg/ml streptomycin at 37° C. in a humid incubator. 293F cells (Invitrogen, Paisley, UK) were grown in serum-free Freestyle medium (Invitrogen) in an orbital shaker at 50 rpm at 37° C. Where appropriate, cells were selected in 1 mg/ml G418 (Invitrogen) or 2 μg/ml puromycin (Sigma-Aldrich, Poole, UK).

### Virus Production

Coxsackievirus was produced as described¹⁸ with modification. Plasmid eGFP-CVB3 encoding strain pH3 with an eGFP sequence and a cleavage sequence at the N-terminus of the viral polypeptide was transfected into a 10 cm dish of HEK293T cells using Superfect (Qiagen, Crawley, UK) according to manufacturer's instruction. After 48 h, cells were mechanically dislodged from the dish, freeze-thawed three times to release virions, and supernatant clarified at 1,000 g before filtration at 0.45 μm. Viral stock was expanded in HeLa cells for 48 h, virus particles harvested by freeze-thaw and filtration as above. Aliquots were frozen at −80° C. until required. Titres were typically in the range of 10⁵ to 10⁷ IU/ml. Adenovirus Ad5-GFP¹⁹ was grown in transcomplementation cell line 293F for 72 h, before three rounds of freeze-thaw to release virus particles and filtration at 0.45 μm. Virus stock was purified by two rounds of ultracentrifugation banding on a caesium chloride gradient, dialysed into PBS/10% glycerol and frozen at −80° C. until required. Titres of purified virus were typically 10⁸ to 10⁹ IU/ml.

### Generation of Stable Knockdown and Over-Expressing Cell Lines

Human TRIM21 DNA was cloned into pDONAI (Takara, Saint-Germain-en-Laye, France) as a NotI/SalI restriction fragment to generate pDON-T21. DNA encoding a small hairpin (sh) RNA directed to human TRIM21 sequence GCAGCACGCTTGACAATGA was cloned into pSIREN Retro-Q (Clontech) to produce pSIREN-shT21. Control shRNA directed to luciferase was encoded by pSIREN-shLuc. Retroviral transduction particles were produced by transfection of 4×10⁶ HEK293T cells with 5 μg of pDON-T21, pSIREN-shT21, empty pDONAI or pSIREN-Luc along with 5 μg each of MLV gag-pol expression plasmid pCMVi and VSV-G expression plasmid pMDG²⁰. Supernatant was harvested after 72 h and filtered at 0.45 μm and used to transduce HeLa cells. Stably transduced cells were selected with G418 (pDON-T21, pDONAI) or puromycin (pSIREN-shT21, pSIREN-shLuc). Levels of TRIM21 protein were monitored by western blotting (sc-25351, Santa Cruz).

Transient siRNA Knockdown

Cells were plated at 1×10⁵ cells per well in six-well plates and allowed to adhere overnight. 150 pmol each of small interfering (si) RNA oligonucleotides T21siRNA1 (UCAUUGUCAAGCGUGCUGC; Dharmacon, Lafayette, Colo., USA) and T21siRNA2 (UGGCAUGGAGGCACCUGAAGGUGG; Invitrogen) or 300 pmol control oligo (Invitrogen) were transfected into cells using Oligofectamine (Invitrogen). Cells were washed after 3 h and incubated for 72 h before infection. Where indicated, 1000 U IFN-α (PBL InterferonSource, Edison, N.J., USA) was added 48 h after knockdown.

### Virus Neutralisation Assays

For both Ad5-GFP and eGFP-CVB3 infections, target HeLa cells were seeded at 1×10⁵ cells per well in 2 ml complete DMEM in six-well plates the day before infection. Where stated, cells were incubated with 1000 U IFN-α. 5×10⁴ infectious units (IU) AdV5-GFP were incubated with antibody in a 10 μl volume for 30 min at room temperature before addition to cells. Cells were incubated for 48 h before washing, trypsinisation and fixing in 4% paraformaldehyde. For coxsackievirus, 2×10⁴ IU were incubated with antibody in a 200 μl incubation for 30 min at room temperature. Infected cells were fixed 8 h after infection to preclude spreading infection. For both viruses, GFP positive cells were enumerated by flow cytometry (FACSCalibur, BD Biosciences, San Jose, Calif., USA).

Antibodies used in VNAs were pooled human serum IgG and IgM (090707 and 090713; Athens Research and Technology, Athens, Ga., USA), purified 9C12 anti-adenovirus 5 hexon mouse IgG (hybridoma obtained from the Developmental Studies Hybridoma Bank, University of Iowa, Iowa, USA), goat anti-adenovirus polyclonal antibody (0151-9004, Abd Serotec, Oxford, UK and AB1056, Millipore, Watford, UK).

### Immunofluorescence

2.5×10⁴ HeLa cells were seeded onto coverslips in 24-well plates and allowed to adhere overnight. Cells were washed twice in DMEM before infection. 5×10⁴ IU AdV5-GFP were incubated with polyclonal or monoclonal anti-hexon adenovirus antibody (eg. 500 ng of mouse monoclonal IgG in a 20 μl volume for 30 min at room temperature before addition of 230 μl DMEM). Cells were infected with 250 μl of this mixture for 30 min at 37° C. Cells were washed three times with PBS, fixed with 4% paraformaldehyde, permeabilised with 0.5% Triton X-100 in PBS and blocked with PBS-BSA (5% bovine serum albumin, 0.1% Tween in PBS) for 1 h. Immunostaining for TRIM21 was performed with a rabbit 50 kDa Ro/SSA primary antibody 20960 (Santa Cruz Biotechnology, Inc., Santa Cruz, U.S.A.) and for ubiquitin with a goat primary 6085 (Santa Cruz Biotechnology, Inc., Santa Cruz, U.S.A.) at 1 in 200 dilution in PBS-BSA. AlexaFluor-conjugated secondary antibodies (Invitrogen) were used to detect primary antibodies at 1 in 200 dilution. Streptavidin coated 0.25 μm latex beads (Sigma-Aldrich) were incubated with rabbit anti-streptavidin polyclonal serum S6390 (Sigma-Aldrich) overnight at 4° C. Beads were washed three times with PBS and transfected into cells using Oligofectamine. Cells were washed with PBS 3 h after transfection and fixed as above. Immunostaining for TRIM21 was performed with immune serum raised in mouse against recombinant TRIM21 RBCC and for conjugated ubiquitin as above both at 1 in 200 dilution in PBS-BSA. AlexaFluor-conjugated secondary antibodies (Invitrogen) were used to detect primary antibodies at 1 in 500 dilution. Confocal images were taken using a Zeiss 63× lens on a Jena LSM 710 microscope (Carl Zeiss Microlmaging GmbH, Germany).

### Fate-of-Capsid Assay

HeLa cells were plated at 2×10⁵ cells per well in a 6 well plate in 2 ml DMEM and left overnight to attach. A proportion of the wells were treated with 8 μM MG132 (Boston Biochem) for 4 h. Untreated cells were exposed to an equivalent quantity of DMSO for the duration of the treatment. 4×10⁷ IU Ad5-GFP were mixed with 6 μg 9C12 monoclonal antibody and incubated at room temp for 30 min then added onto the cells in 1 ml complete media. Infections were incubated at 37° C. for 1 hr before removing infection mixtures and replacing with DMEM. Cells were harvested at indicated time points post initial infection and boiled in 100 μl 1×LDS sample buffer with reducing agent (Invitrogen). Virus was detected with goat anti-hexon Ad5 (1:1000, AB1056, Millipore) and HRP conjugated anti goat IgG (1:5000, sc-2056, Santa Cruz). Antibody was detected with donkey anti-mouse IgG (1:500, AP192 Millipore) and protein A-HRP (1:2000, 610438, BD Biosciences). TRIM21 was detected with TRIM21 RBCC immune sera (1:2000) and protein A HRP to avoid cross-reaction to the mouse antibody on the gel.

### Immunoblotting

Cells from a single well of a 6 well plate were scraped off, resuspended and heated at 98° C. for min in 100 μl 1×LDS sample buffer with reducing agent (Invitrogen). Equal volumes were loaded onto a 4-12% NuPAGE gel and electrophoresed in 1×MOPS buffer (Invitrogen). Proteins were transferred onto Protran nitrocellulose membrane (Whatman) and immunoblotted with the indicated antibodies. In all cases blots were incubated with antibody in PBS containing 5% milk, 0.1% Tween and washed with PBS-Tween. Visualisation was carried out using ECL Plus Western Blotting Detection System (GE Healthcare). Westerns were stripped for re-probing as per manufacturers instructions with 1× Re-Blot Plus Strong Solution (2504, Millipore). Loading control blots were carried out with rabbit polyclonal β-actin (1:1000, #4967, Cell Signalling).

### Fluorescence Titration

Full-length and ΔRING-Box recombinant TRIM21 was expressed as MBP-fusion proteins in E. coli and purified using amylose resin and size-exclusion chromatography. The MBP tag was removed via tev protease cleavage and cleaved TRIM21 was dialysed into 20 mM Tris pH8, 100 mM NaCl, 1 mM DTT. Steady-state fluorescence titration experiments were performed at 20° C. using a Cary Eclipse fluorescence spectrophotometer (Varian) with excitation at 296 nm and emission at 335 nm, using 15 nm slit-widths and a PMT voltage of 850. The quenching in intrinsic TRIM21 tryptophari fluorescence upon titration of IgG was measured with an averaging time of 5 s. Each titration was fit using Kaleidagraph (Synergy Software) to the quadratic expression F=F_{TR}+f′(−(I₀−TR₀+K_{d})±(((I₀−TR₀+K_{d})²)+(4K_{d}TR₀))1/2))/2; where F is the observed fluorescence, F_{TR} is the molar TRIM21 fluorescence, f′ is the molar change in fluorescence, (TR₀) is the total TRIM21 concentration, (I₀) is the total antibody concentration, and K_{d} is the dissociation constant.

### Fluorescence Anisotropy

The PRYSPRY domain of TRIM21 was expressed and purified as previously described^{4,5}. The protein was labelled with Alexa Fluor 488 5-SDP ester (Invitrogen) and dialysed into 50 mM Tris pH 8 with 200 mM NaCl. Anisotropy experiments were performed using a Cary Eclipse fluorescence spectrophotometer (Varian) with excitation at 488 nm and emission at 530 nm, using 10 nm slit-widths and a PMT voltage of 600. IgM (Athens Research and Technology, Athens, Ga., USA) was titrated into 50 nM PRYSPRY and the polarised fluorescence averaged over 5 s. The dissociation constant (KO was determined by fitting the change in anisotropy to the quadratic expression given above using Kaleidagraph (Synergy Software).

### SEC MALS

SEC MALS was performed using a Wyatt Heleos II 18 angle light scattering instrument coupled to a Wyatt Optilab rEX online refractive index detector. Samples were prepared as described above and resolved on a Superdex S-200 analytical gel filtration column running at 0.5 ml/min before passing through the light scattering and refractive index detectors in a standard SEC MALS format. Protein concentration was determined from the refractive index based on 0.186 ΔRI for 1 mg/ml, and combined with the observed scattered intensity to calculate absolute molecular mass using Wyatt's ASTRA analysis software. The major species in TRIM21 has a mass of 107 kDa averaged across the indicated region of the peak. The predicted mass of monomeric TRIM21 is 54 kDa, making TRIM21a dimer in solution and not a trimer as previously reported. SEC MALS of IgG gives the expected mass of 154 kDa with small (<10%) levels of dimer mass 325 kDa, which is typical for IgG. TRIM21-IgG complex resolves as multiple peaks, corresponding to excess IgG with mass and elution volume as previously and a peak with mass ˜280 kDa. The 280 kDa peak is consistent with a 1:1 complex of TRIM21:IgG, where each protein is a homodimer.

### Complementation Neutralisation Assay Using Exogenous TRIM21

HeLa cells were seeded at 1×10⁵ cells per well in 2 ml complete DMEM in six-well plates the day before infection. 5×10⁴ IU AdV5-GFP were incubated with 4 μg of goat anti-adenovirus polyclonal antibody (AB1056, Millipore, Watford, UK) for 15 min before addition of 200 μg of appropriate recombinant TRIM21 protein, 100 μl total volume, and incubation for a further 15 min at room temperature. Media on the cells were exchanged for this mixture made up to 1 ml with complete DMEM. Cells were incubated at 37° C. in a humid incubator for 48 h and then treated as in a virus neutralisation assay (see above).

### In Vitro Ubiquitination Assays

In vitro assays were carried out largely as described²¹. Reactions were carried out in 1× Ubiquitination buffer (50 mM Tris-HCl pH7.4, 2.5 mM MgCl₂, 0.5 mM DTT) with the addition of 2 mM ATP, 300 ng His-Ubal, 300 ng His-UbCH5c, 1 μg ubiquitin (Sigma) and 50 ng MBP-TRIM21 or MBP-TRIM21 ΔRing-Box as indicated. Human Ubal and UbCH5c were expressed in bacteria and purified using Ni-NTA resin (Qiagen) as described²¹. Antibody adenovirus mixtures were made by incubating 5×10⁴ IU AdV5-GFP per 150 ng goat polyclonal anti-hexon (Millipore) for 30 min, where 1 μl mix contains 3.6×10⁴ IU and 106 ng antibody. Increasing amounts were added into the reaction mixture as indicated. Controls with either just Ad5 or anti-hexon antibody contained 1.25×10⁵ IU and 150 ng antibody respectively. Reaction mixtures were incubated at 37° C. for 1 h then stopped by addition of LDS sample buffer and heating to 98° C. for 5 min. Samples were run on a gel and Western blotted for TRIM21 (1:500, sc-25351, Santa Cruz), Ad5 hexon (donkey anti-goat IgG HRP 1:5000 sc-2056, Santa Cruz) or ubiquitin (1:1000, FK-2, Enzo Life sciences) as indicated.

### Example 1

### Antibodies are Internalised

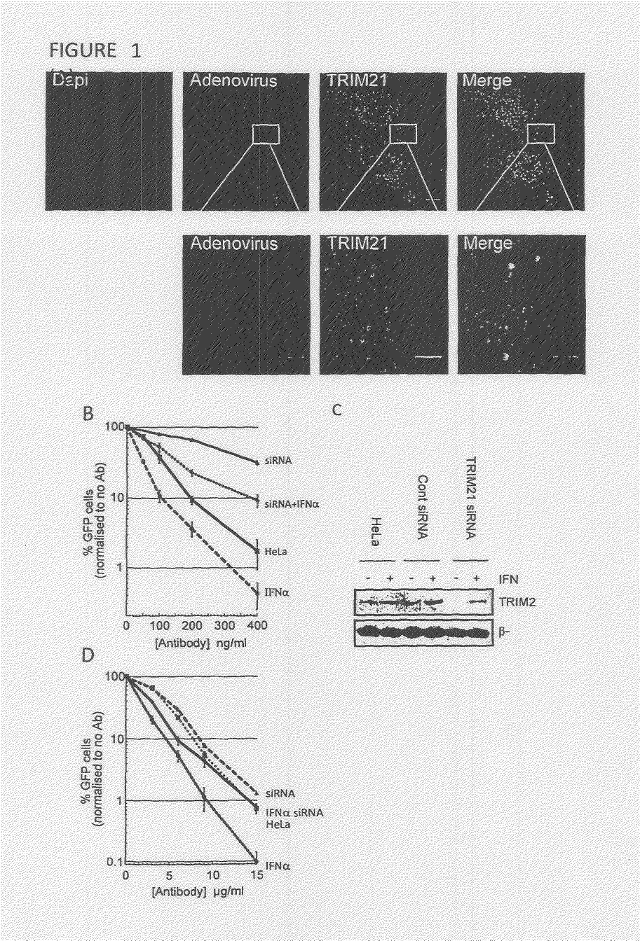

It is assumed that antibodies do not routinely enter the cytosol during viral infection. To test this, Applicants pre-incubated adenovirus (a model human virus that causes respiratory disease) with antibody and added the virions to cultured HeLa cells. Adenovirus was chosen as it is a non-enveloped virus and its capsid is naturally exposed to serum antibody prior to cellular infection. After 30 minutes of infection the cells were fixed and a fluorescent anti-IgG antibody was added to detect antibody-coated virions. As can be seen in FIG. 1A, antibody-coated virions successfully infect cells. Similar results were obtained using polyclonal anti-hexon antibodies and human serum IgG. Adenovirus enters the cell by binding to the CAR receptor and becoming endocytosed. Applicants found that addition of antibody does not prevent this process and that antibody remains attached to virus post-entry.

### Example 2

### TRIM21 Mediates Intracellular Viral Neutralisation

To address whether antibody-coated virus is accessible to cytosolic TRIM21, Applicants co-stained for TRIM21. As shown in FIG. 1A, TRIM21 is efficiently recruited to antibody-coated viral particles.

Next, Applicants tested the effect of TRIM21 recruitment to virions by quantifying the levels of adenovirus infection. Applicants used a virus that carries a GFP gene so that infection efficiency could be determined by flow cytometry analysis. A standard viral neutralisation assay was performed on HeLa cells pre-treated with control siRNA, TRIM21 siRNA, interferon-α (IFNα) or IFNα and TRIM21 siRNA (FIG. 1B). To take account of toxicity and variable cell death between these different conditions, Applicants measured the decrease in infection due to the addition of antibody. In the absence of antibody, adenovirus infected ˜50% of cells. The percentage of infected cells decreased rapidly with increasing antibody concentration such that at 400 ng/μl antibody, infection was reduced by >50-fold (FIG. 1B). However, in cells depleted of TRIM21, addition of 400 ng/μl antibody had a minimal effect on infection (˜3-fold).

During an immune response, IFNα activates the transcription of antiviral genes. Applicants found that TRIM21 is IFNα regulated and that the modest levels of endogenous TRIM21 protein are greatly increased by IFNα (FIG. 1C). Consistent with this result, pre-incubation of cells with IFNα increased the effect of antibody neutralisation such that at 400 ng/μl antibody, infection decreased >230-fold. IFNα has pleiotropic effects but without addition of antibody Applicants observed little impact on adenovirus infection. To show that IFNα/antibody neutralisation synergy is TRIM21-dependent, Applicants specifically depleted the TRIM21 levels that are up-regulated by IFNα, leading to >95% recovery of infectivity (FIG. 1B). In all experiments, antibody neutralisation of viral infection directly correlated with TRIM21 levels (FIG. 1C). For example, cells expressing the most TRIM21 were almost 2 orders of magnitude more resistant to adenovirus infection than those expressing the least.

### Example 3

### Variation of Conditions

To demonstrate that TRIM21/antibody intracellular neutralisation is not adenovirus-specific, Applicants tested its effect on coxsackievirus B3 infection. Coxsackievirus B3 is a picornavirus, of the same genus as poliovirus, and is a leading cause of aseptic meningitis. A replication-competent strain bearing a GFP-reporter gene was used to infect HeLa cells pre-treated with combinations of TRIM21 siRNA and IFNα as described above. Infection time was limited to <16 hrs to prevent spreading infection. Endogenous levels of TRIM21 were insufficient to mediate a significant block (infection increases 2-fold upon TRIM21 depletion), however, treatment with IFNα gave an almost total block to infection in the presence of 15 μg/ml antibody (FIG. 1D). Depletion of TRIM21 in IFNα-treated cells recovered infection levels to those of untreated cells, demonstrating that this is a TRIM21-dependent effect.

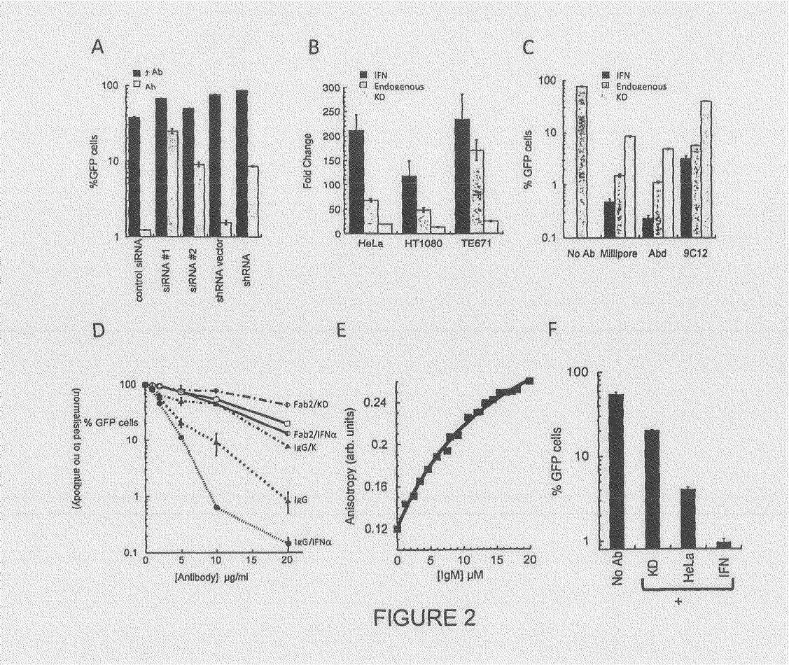

Applicants confirmed the robustness of this phenotype by examining the effect of different siRNA sequences, cell types and types of antibody. As can be seen in FIG. 2A, different TRIM21 siRNA's with different target sequences reversed antibody neutralisation of adenovirus infection by knocking-down TRIM21 levels. Next, Applicants tested a range of cell lines including HeLa, HT1080 and TE671. A stable TRIM21 knockdown line was established in each case using an shRNA vector based on the sequence of siRNA 2. In all cells, TRIM21 mediated antibody neutralisation of adenovirus (FIG. 2B). Finally, Applicants tested the effect on adenovirus neutralisation of two different anti-Ad5 polyclonal antibodies (Abd Serotec and Millipore) and an anti-Ad5 hexon monoclonal (9C12). In every case, neutralisation of adenovirus was enhanced by TRIM21 upregulation and reversed by TRIM21 KD (FIG. 2C).

It is commonly thought that antibodies neutralise virus by blocking receptor binding and preventing cell entry. However, >90% of the antibody neutralisation of adenovirus Applicants observe is mediated by TRIM21 (for example, at 200 ng/μl antibody there are 0.27% infected cells in HeLa controls versus 10% in cells depleted of TRIM21). To test which of these two mechanisms dominates in a polyclonal response, Applicants looked at the neutralisation of adenovirus by pooled human serum IgG. Applicants found that the majority of the neutralisation affect (within the concentration range tested) was mediated by TRIM21 (FIG. 2D). TRIM21 binds to IgG via the Fc domain, therefore antibody fragments lacking the Fc should no longer be capable of neutralising virus as effectively. To confirm that it is TRIM21 and not receptor blocking that is the primary source of viral neutralisation Applicants treated serum IgG with pepsin. Pepsin cleavage of IgG removes the Fc and generates Fab2 fragments, which are still bivalent and capable of cross-linking antigen. Moreover, Fab2 fragments bind antigen with the same affinity as IgG. Applicants found that Fab2 fragments were no longer able to neutralise adenovirus infection efficiently (FIG. 2D). Furthermore, when using Fab2, IFNα treatment or TRIM21 KD no longer affected adenovirus infection.

### Example 4

### Interaction with IgM and IgA

The foregoing exampled demonstrate that in order for antibodies to mediate intracellular viral neutralisation they must contain an Fc-fragment and TRIM21 must be present.

During the early stages of infection, in which innate immunity is critical, IgM rather than IgG antibodies dominate the antibody repertoire. Applicants tested whether TRIM21 interacts with IgM and if so the importance of TRIM21 in IgM viral neutralisation. To investigate TRIM21:IgM binding, Applicants labelled the TRIM21 PRYSPRY domain with an Alexa 488 fluorophore and measured its fluorescence anisotropy upon titration of IgM (FIG. 2E). The resulting titration curve was fit (Materials & Methods) to give an affinity (K_{D}) of 16.8 μM±1.5 μM. The in vivo affinity of TRIM21 to IgM is likely to be significantly higher however as full-length TRIM21 is a multimer. Complement C1q, which binds IgM with nanomolar affinity, has undetectable affinity when measured as a monomer⁶.

Next, Applicants tested the effect of serum IgM on adenovirus infection. Applicants found that pooled human serum IgM and TRIM21 operate synergistically to neutralise adenovirus infection (FIG. 2F). Furthermore, as with IgG, the neutralisation of virus by IgM required TRIM21. This suggests that TRIM21 works alongside innate immunity and is protective in the early stages of a humoral immune response.

The same effect was seen in respect of IgA (FIG. 2G). IgA is important as it is the major isotype in the mucosa, which is often the first point of contact with a virus. An infection experiment using serum secreted IgA shows that TRIM21 can use IgA to neutralize virus. Anti-TRIM21 siRNA prevents viral neutralisation, whilst IFN-α potentiates it.

### Example 5

### Mechanism of TRIM21 Immunity

The previous examples demonstrate that there is an intracellular immune response mediated by TRIM21 and antibodies that is capable of preventing viral infection. Next, Applicants examined the mechanism by which this intracellular neutralisation occurs. Applicants investigated the mechanism in three ways. First, Applicants determined how TRIM21 targets antibody and the thermodynamics of interaction. Second, Applicants examined what events subsequent to targeting are required for neutralisation. Third, Applicants asked how virus is neutralised.

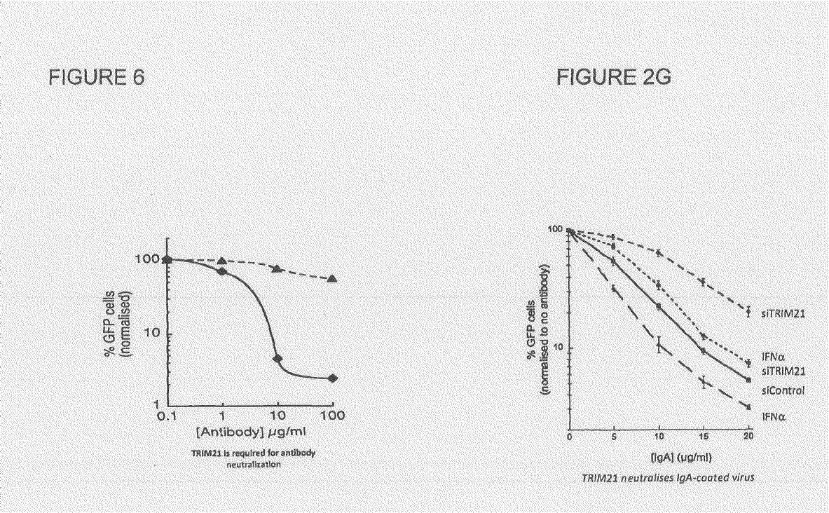

TRIM21 is a multi-domain protein consisting of RING, B Box, coiled-coil and PRYSPRY domains. Applicants tested the role of these domains in IgG binding using multi-angle light scattering (MALS) and fluorescence titration spectroscopy. Analysis of the MALS data reveals that recombinant full-length TRIM21 forms a stable dimer and not a trimer as previously reported⁷ (FIG. 3A). Furthermore, when mixed with IgG, TRIM21 forms a stoichiometric complex consisting of 1 antibody and 1 TRIM21 (FIG. 3A). Deletion of the RING domain alone resulted in a destabilised recombinant protein, however deletion of both RING and B Box did not affect TRIM21 stability or its ability to dimerise (data not shown). Fluorescence titration spectroscopy revealed that full-length TRIM21 and ΔRING-Box bound to IgG with a similar dissociation constant (K_{D}) of <1 nM (FIGS. 3B&C). As the monomeric PRYSPRY domain binds with ˜150 nM affinity^{4,5}, this indicates that the coiled-coil domain is required for TRIM21 dimerisation and the simultaneous engagement of both IgG heavy chains. The sub-nM affinity of TRIM21 for IgG makes TRIM21 the highest affinity antibody receptor in the human body. The evolution of such a high affinity interaction explains how TRIM21 efficiently targets virus.

Next, Applicants looked at what happens to the virus after TRIM21 is recruited and the role of the RING and B box domains. As RING domains often display E3 ubiquitin ligase activity, Applicants hypothesised that TRIM21 may target bound virus for degradation via ubiquitination. Cells possess two pathways for degradation of ubiquitinated material—the proteasome and autophagy. To explore the role of these pathways in TRIM21 neutralisation of virus Applicants performed viral infection experiments in the presence of MG132 (a proteasome inhibitor) and 3-methyladenine (3-MA; an autophagy inhibitor). The autophagy inhibitor had no affect on infectivity, however MG132 significantly reversed TRIM21 neutralisation of infectivity (FIG. 3D). Titration experiments showed a direct correlation between increasing levels of MG132 and reduced neutralisation (FIG. 3E). The ability of MG132 to reverse neutralisation was dependent upon the presence of antibody and TRIM21. Moreover, addition of MG132 could not recover infection in cells depleted of TRIM21, showing that TRIM21 and proteasome function are essential components on the neutralisation pathway (FIG. 3F).

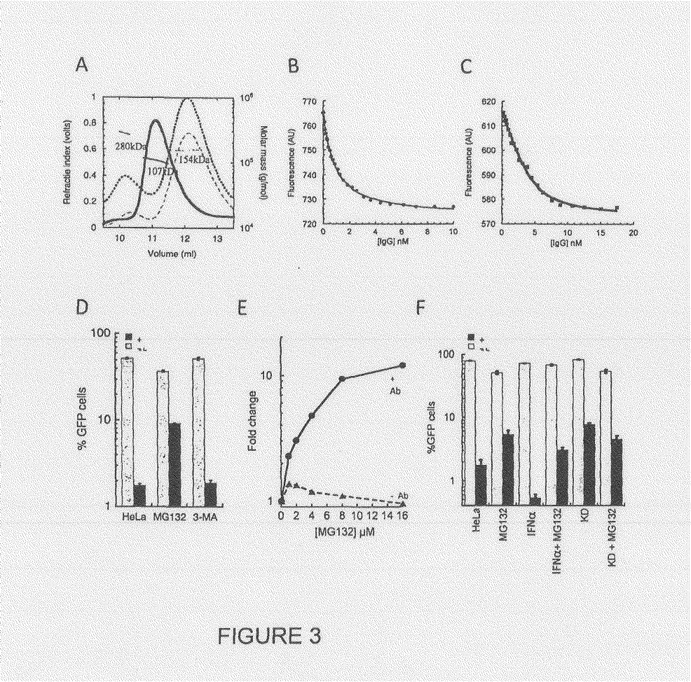

To determine whether ubiquitination is essential to target virus to the proteasome, Applicants tested the ability of full-length TRIM21 and ΔRING-Box recombinant proteins to neutralise infection. Applicants incubated protein with antibody-coated virions and allowed the virus to infect cells depleted of TRIM21. As can be seen in FIG. 4A, deletion of the RING and B Box domains prevents TRIM21 neutralisation of virus. Applicants attempted to repeat these experiments in cells over-expressing TRIM21, however this led to loss of function. Loss of function could be partially reversed with interferon suggesting that over-expression titrates essential co-factors rather than generates inactive protein (data not shown). To confirm that the neutralisation Applicants observe with recombinant proteins correlates with ubiquitin ligase activity, Applicants compared the ability of full-length and ΔRING-Box proteins to auto-ubiquitinate. Whilst deletion of the RING and B Box domains has no effect on IgG binding, it abolishes ubiquitination (FIG. 4B). Thus both TRIM21 ubiquitination activity and proteasomal function are required for viral neutralisation. To demonstrate that it is the intracellular TRIM21-associated viral particle that is ubiquitinated, Applicants examined infected cells by confocal microscopy and stained for ubiquitin. As can be seen in FIG. 4C, virions co-localised with TRIM21 are also positive for ubiquitin.

Whilst E3 ubiquitin ligases are known to auto-ubiquitinate, it is the transfer of ubiquitin to substrate that is thought to be important for proteasomal targeting. However, proteasomal targeting via auto-ubiquitination would allow TRIM21 to neutralise any virus and prevent evolution of viral mutants that escape ubiquitin-conjugation. Consistent with this mechanism, whilst Applicants found that TRIM21 efficiently forms ubiquitin chains on itself. Applicants found no detectable ubiquitination of either IgG or virus in Applicants' in vitro ubiquitination assay (FIG. 4D). This correlates with the extremely high affinity with which TRIM21 has evolved to bind antibody. If TRIM21 were transferring ubiquitin to antibody or virus through normal enzymatic turnover then a high affinity would translate as a highly inefficient K_{M}.

To determine what happens to virus after TRIM21-mediated targeting to the proteasome, Applicants performed a fate-of-capsid timecourse experiment. Applicants compared the levels of hexon protein (viral capsid) in infected HeLa cells to those in cells depleted of TRIM21. By 2 hours post-infection there was markedly less hexon in HeLa compared to TRIM21 depleted cells (FIG. 4E). This indicates both that TRIM21 mediates degradation of virus and that it is a rapid process. Addition of MG132 prevented the decline in hexon levels, confirming that virus is being physically degraded in a proteasome-dependent manner. As proteasomal targeting by TRIM21 requires virus to be antibody-bound, Applicants also looked at the antibody levels in infected cells. Applicants found that the destruction of virus is paralleled by disposal of antibody (FIG. 4E). In contrast, Applicants saw little affect of MG132 on TRIM21 levels suggesting that only a fraction of the total pool of cellular TRIM21 is degraded or that TRIM21 is recycled (FIG. 4E).

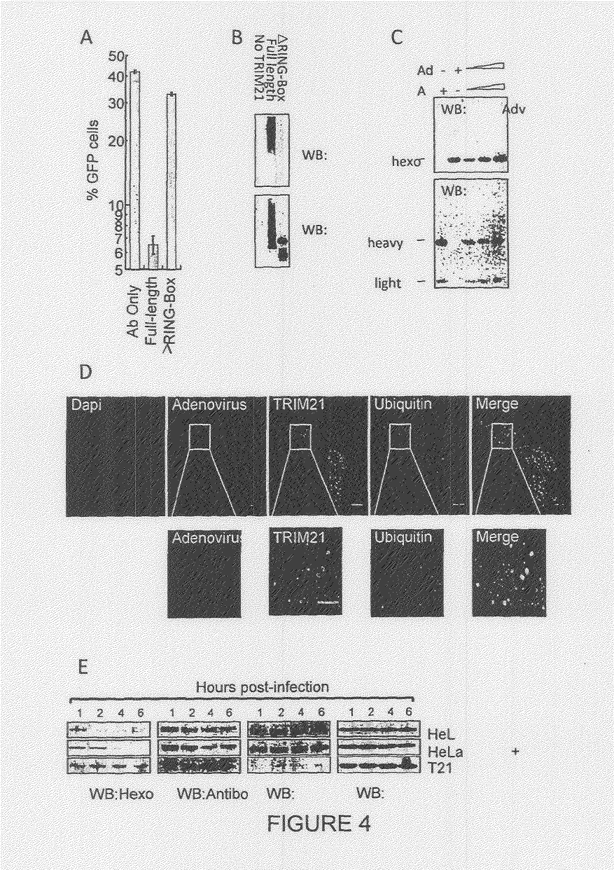

The combination of antibody targeting and TRIM21 auto-ubiquitination mean that no direct viral interactions are required for neutralisation. This means that TRIM21-mediated immunity should be broadly effective against most intracellular pathogens. To test this, Applicants transfected cells with streptavidin latex beads coated in anti-streptavidin antibody. TRIM21 is efficiently recruited to the antibody-bound beads (FIG. 5). Furthermore, TRIM21 associated beads are positive for ubiquitin. Thus, TRIM21 does not require any direct pathogen interaction for binding or ubiquitination, should be effective against a broad spectrum of pathogens and be difficult to evade.

### Example 6

### Activity in Cell Culture

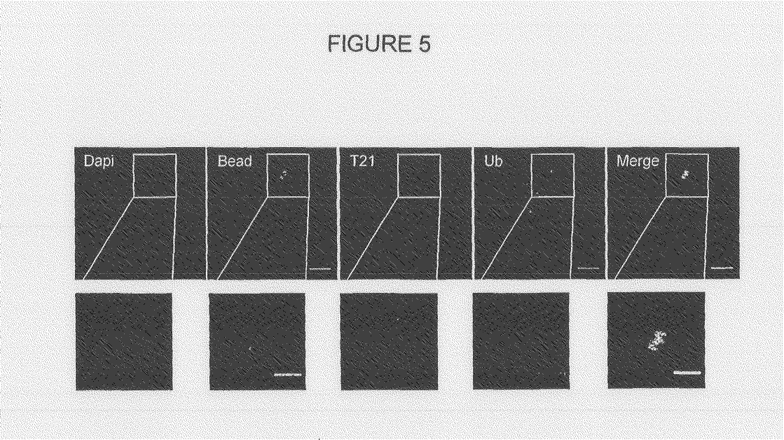

Embryonic fibroblast cells were prepared from either wild-type or TRIM21 KO C57BL/6 mice (22) and challenged with GFP-adenovirus in the presence of 9C12, a monoclonal anti-hexon antibody (available from DSHB, Iowa); hexon is the major coat protein of adenovirus. 9C12 potently prevented infection of cells from wild-type mice but had almost no affect preventing infection of cells from knock-out mice. Almost all the cells from the knock-out were infected even in the presence of saturating concentrations of antibody. See FIG. 6. This shows that TRIM21 provides very potent protection against viral infection and that it is important for the ability of antibodies to neutralize, as a potently neutralizing antibody becomes non-neutralizing in the absence of TRIM21.

### Example 7

### Activity in Wild-Type and Knock-Out Mice

### Virus Preparation

A 3T3 mouse fibroblast cell line was infected with mouse adenovirus type 1 (MAV-1) reference strain purchased from American Type Culture Collection (ATCC). Four days later infected cells and supernatant were collected. Virus was released from the cells by 3 repeated freeze-thaw cycles. Cell supernatant and pellet free cell lysate were pooled together and MAV-1 particles were purified by twice by equilibrium centrifugation in continuous CsCl gradients. Virus was quantified by measuring A₂₆₀ value which corresponded to 1.8×10¹³ pfu/ml. Virus infectivity was measured by end point dilution assay and tissue culture 50% infectious dose value (TCID50), calculated by the Reed and Munch method, was 8.4×10⁸/ml or 5.8×10⁸ pfu/ml.

### Experimental Infections

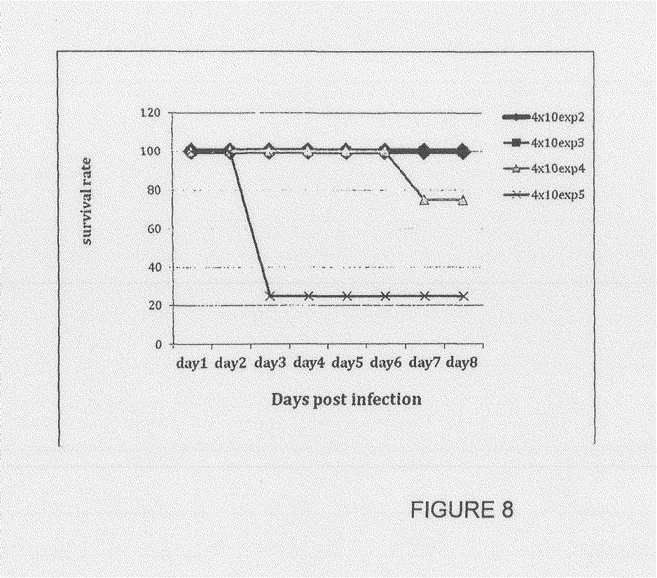

For LD50 determinations, six-week-old C57BL6 mice were infected by intraperitoneal (i.p.) injection (four animals per dose) of 10-fold serially diluted doses of MAV-1 in 100 ul of PBS and observed up to twice daily for morbidity and mortality. Infection of wild type mice with 4×10⁵ pfu resulted in 75% mortality rate (FIG. 8). Therefore for all further experiment in C57BL/6 wild type and TRIM21 knockouts mice Applicants choose a subclinical 4×10⁴ pfu dose of MAV-1.

### Viral Titres

To test involvement of TRIM21 in immunity to infection Applicants challenged 6 WT and 6 KO naïve mice with 4×10⁴ pfu dose of MAV-1. Unless mice exceed moderate symptoms they were culled on day 9 p.i. Spleen and brain were collected from culled animals and both virus and genomic DNA were prepared. Genomic DNA was used in RT-PCR with specific hexon primers to quantitate viral levels. Virus was titrated by TCID50 to determine viraemia in each animal. The experiment was designed such that the ability of TRIM21 to augment the primary immune response (IgM) is the principle determinant of survival and/or viraemia.

To determine the role of TRIM21 in protective immunity (IgG) the experiment was performed on MAV-1 challenged mice that have neutralizing antibody to the virus. Therefore, mice were challenged with a subclinical dose of MAV-1, and rechallenged after 9 days with a clinical dose of virus.

In both experiments, it is observed that TRIM21 KO mice show increased viral load and/or mortality as a result of MAV-1 infection. Applicants conclude that the presence of TRIM21 is important for mediating the antiviral effects of antibody treatment in mouse, which are known to be effective against adenovirus infection (16).

### Example 8

### Antibody-TRIM21 Fusion

Applicants have tested a monoclonal anti-hexon mouse antibody (9C12) and found it to possess potent neutralization activity against adenoviral infection (see Example 6). cDNA was prepared from the 9C12 hybridoma cells and light and heavy chains were amplified by PCR using the following primers:

LIGHT CHAIN
Kappa chain into CMV promoter of pBudCE4.1:
FORWARD:
VL1S
acgtGTCGACccaccATG GAG ACA GAC ACA CTC CTG CTA T
VL2S
acgtGTCGACccaccATG GAT TTT CAA GTG CAG ATT TTC AG
VL3S
acgtGTCGACccaccATG GAG WCA CAK WCT CAG GTC TTT RTA
VL4S
acgtGTCGACccaccATG KCC CCW RCT CAG YTY CTK GT
VL5S
acgtGTCGACccaccATG AAG TTG CCT GTT AGG CTG TTG
REVERSE
CLX
catgtctagaCTAACACTCATTCCTGTTGAAGC
HEAVY CHAIN
Heavy chain gamma-1 into EF-1a promoter of
pBudCE4.1 (FIG. 7):
FORWARD
VH1 N
aataGCGGCCGCcaccATGGRATGSAGCTGKGTMATSCTCTT
VH2N
aataGCGGCCGCcaccATGRACTTCGGGYTGAGCTKGGTTTT
VH3N
aataGCGGCCGCcaccATGGCTGTCTTGGGGCTGCTCTTCT
VH4N
aataGCGGCCGCcaccATGATRGTGTTRAGTCTTYTGTRCCTG
REVERSE
CHKpn
catgGGTACCTCATTTACCAGGAGAGTGGGAG
Where: r = a, g; y = c, t; m = a, c; k = g, t;
s = c, g; w = a, t; v = a, c, g; n = a, c, g, t.

Amplified DNA was then sequenced to give the following light and heavy chain sequences:

Heavy Chain: SEQ ID NO 13

Light Chain: SEQ ID NO 14

The corresponding amino acid sequence was then reverse translated into a codon optimised DNA sequence for expression in pBudCE4.1 (FIG. 7), Each chain was cloned with an N-terminal secretory signal to ensure secretion of the antibody protein.

The optimised sequences are as follows:

Heavy Chain: SEQ ID NO 15

Light Chain: SEQ ID NO 16

The resulting recombinant 9C12 expression vector was then used as the starting point to clone fusion proteins, such that the C-terminus of the heavy chain (end of the C_{H}3) was joined via a short linker to the beginning of TRIM21. Three variants were cloned representing fusion of either full-length TRIM21, the RING, B Box and Coiled-coil domains, or the RING and B Box. In one form, the resulting fusion sequences were:

9C12-FuII TRIM21 fusion: SEQ ID NO 17

9C12-RBCC fusion: SEQ ID NO 18

9C12-RB fusion: SEQ ID NO 19

In each case these heavy chain fusions were expressed together with an unmodified light chain in the multi-chain expression vector pBud (see FIG. 7). Expression of these constructs can be performed in cell lines such as the suspension cell line 293 F. Antibody is secreted into the medium. In order to purify antibody from media, supernatant is applied to a Protein A affinity resin and then eluted in low pH buffer. After elution, the purified protein is returned to a physiological saline buffer. The purity of purified protein is assayed by SDS PAGE.

The following experiments are then used to test the efficacy of the chimeric proteins: GFP adenovirus is pre-incubated with the chimeric proteins at a range of concentrations. The adenovirus-chimera mixture is added to cultured cells at a viral titre designed to yield an MOI of ˜0.5. Infected cells are incubated for ˜24 hours and the infection efficiency determined by FACS analysis by counting the number of GFP positive cells. Cell lines that can be used to test efficacy include adenovirus-permissive cell lines such as 293, HeLa and MEF. To further demonstrate the efficacy of the chimeras, these experiments can be carried out under conditions of endogenous TRIM21 depletion by siRNA or shRNA or in cells where TRIM21 has been genetically knocked-out.

### Example 9

The above example pertains to molecules in which the activities of virus binding and TRIM21 function are combined in a single polypeptide. If this single polypeptide requires another polypeptide chain to be functional (for instance a light chain) this must be included prior to incubation with virus, usually during expression. In the next example Applicants describe a molecule that can bind antibodies and has TRIM21 activity in the absence of endogenous TRIM21 protein. Thus instead of having a single molecule with both virus binding and TRIM21 function, the two activities are separated into two discrete molecules—one with the ability to bind a pathogen (as exemplified by an antibody) and a second with the ability to bind the first and cause the pathogen to be neutralized (as exemplified by TRIM21). Applicants have already described previously the addition of TRIM21 exogenously and shown that this has antiviral activity. This example describes the fusion of the antibody binding domain of Protein A (pA) to the catalytic domains of TRIM21. Three examples are given, using either the RING, B Box and coiled-coil domains, the RING and B Box domains or the RING domain. A further example is a modification in which the Protein A domain is found at the C-terminus. Further constructs are envisaged in which pA is replaced with another antibody binding domain (eg Protein G, selected peptide ligands) and/or in which the catalytic domains are replaced (eg with those of another TRIM protein) to preserve ubiquitin-proteasome recruitment.

pA-RBCC SEQ: ID NO 20

pA-RB: SEQ ID NO 21

pA-R: SEQ ID NO 22

RBCC-Pa: SEQ ID NO 23

These sequences were cloned into a bacterial expression construct with affinity tags to allow efficient purification (His and MBP tags). The proteins were expressed overnight at 25° C., the cells lysed and protein purified on affinity resin followed by gel filtration. Antiviral efficacy was tested by incubating the proteins at a range of concentrations with an antiviral antibody (eg 9c12) and adding the mixture to virus (eg GFP adenovirus) before infecting cultured cells at an MOI of ˜0.5. Permissive cell lines must be used (eg for adenovirus—HeLa, 293, MEFs). Infection efficiency is then determined by FACS, by counting the number of GFP positive cells.

### REFERENCES

1. Murphy K, T. P., Walport, M. Immunobiology, 887 (Garland Science, New York, 2008).
2. Akira, S., Uematsu, S. & Takeuchi, O. Pathogen recognition and innate immunity. Cell 124, 783-801 (2006).
3. Brenner, S. & Milstein, C. Origin of antibody variation. Nature 211, 242-3 (1966).
4. James, L. C., Keeble, A. H., Khan, Z., Rhodes, D. A. & Trowsdale, J. Structural basis for PRYSPRY-mediated tripartite motif (TRIM) protein function. Proc Natl Acad Sci USA 104, 6200-5 (2007).
5. Keeble, A. H., Khan, Z., Forster, A. & James, L. C. TRIM21 is an IgG receptor that is structurally, thermodynamically, and kinetically conserved. Proc Natl Acad Sci USA 105, 6045-50 (2008).
6. Chen, F. H. et al. Domain-switched mouse IgM/IgG2b hybrids indicate individual roles for C mu 2, C mu 3, and C mu 4 domains in the regulation of the interaction of IgM with complement C1q. J Immunol 159, 3354-63 (1997).
7. Rhodes, D. A. & Trowsdale, J. TRIM21 is a trimeric protein that binds IgG Fc via the B30.2 domain. Mol Immunol 44, 2406-14 (2007).
8. Langford, M. P., Villarreal, A. L. & Stanton, G. J. Antibody and interferon act synergistically to inhibit enterovirus, adenovirus, and herpes simplex virus infection. Infect Immun 41, 214-8 (1983).
9. Burdeinick-Kerr, R., Wind, J. & Griffin, D. E. Synergistic roles of antibody and interferon in noncytolytic clearance of Sindbis virus from different regions of the central nervous system. J Virol 81, 5628-36 (2007).
10. Vrijsen, R., Mosser, A. & Boeye, A. Postabsorption neutralization of poliovirus. J Virol 67, 3126-33 (1993).
11. Osiowy, C. & Anderson, R. Neutralization of respiratory syncytial virus after cell attachment. J Virol 69, 1271-4 (1995).
12. Wetz, K., Willingmann, P., Zeichhardt, H. & Habermehl, K. O, Neutralization of poliovirus by polyclonal antibodies requires binding of a single IgG molecule per virion. Arch Virol 91, 207-20 (1986).
13. Wohlfart, C. Neutralization of adenoviruses: kinetics, stoichiometry, and mechanisms. J Virol 62, 2321-8 (1988).
14. Smith, T. J. et al. Structure of human rhinovirus complexed with Fab fragments from a neutralizing antibody. J Virol 67, 1148-58 (1993).
15. Schlesinger, J. J. & Chapman, S, Neutralizing F(ab′)2 fragments of protective monoclonal antibodies to yellow fever virus (YF) envelope protein fail to protect mice against lethal YF encephalitis. J Gen Virol 76 (Pt 1), 217-20 (1995).
16. Oakes, J. E. & Lausch, R. N. Role of Fc fragments in antibody-mediated recovery from ocular and subcutaneous herpes simplex virus infections. Infect Immun 33, 109-14 (1981).
17. Kishimoto, C., Hiraoka, Y. & Takada, H. Effects of immunoglobulin upon murine myocarditis caused by influenza A virus: superiority of intact type to F(ab′)2 type. J Cardiovasc Pharmacol 43, 61-7 (2004).
18. Feuer, R., Mena, I., Pagarigan, R., Slifka, M. K. & Whitton, J. L. Cell cycle status affects coxsackievirus replication, persistence, and reactivation in vitro. J Virol 76, 4430-40 (2002).
19. de Martin, R., Raidl, M., Hofer, E. & Binder, B. R. Adenovirus-mediated expression of green fluorescent protein. Gene Ther 4, 493-5 (1997).
20. Yee, J. K., Friedmann, T. & Burns, J. C. Generation of high-titer pseudotyped retroviral vectors with very broad host range. Methods Cell Biol 43 Pt A, 99-112 (1994).
21. Mallery, D. L., Vandenberg, C. J. & Hiom, K. Activation of the E3 ligase function of the BRCA1/BARD1 complex by polyubiquitin chains. EMBO J 21, 6755-62 (2002).
22. Yoshimi R, Chang T H, Wang H, Atsumi T, Morse H C 3rd, Ozato K. “Gene disruption study reveals a nonredundant role for TRIM21/Ro52 in NF-kappaB-dependent cytokine expression in fibroblasts.” J. Immunol. 2009 Jun. 15; 182(12):7527-38

The invention is further described by the following numbered paragraphs:

1. A compound comprising:
(a) a ligand which binds, directly or indirectly, specifically to an antigen of a pathogen, provided that said ligand is not the PRYSPRY domain of TRIM21; and
(b) a RING domain and/or an inducer of TRIM21 expression.

2. A compound according to paragraph 1, wherein the ligand binds directly to the antigen and is selected from the group consisting of at least part of an immunoglobulin molecule, a peptide and/or nucleic acid aptamer, and a structured polypeptide ligand.

3. A compound according to paragraph 2, wherein the immunoglobulin molecule is selected from the group consisting of an IgG, IgA, IgM, IgE, IgD, F(ab′)₂, Fab, Fv, scFv, dAb, V_{HH}, IgNAR, a modified TCR, and multivalent combinations thereof.

4. A compound according to paragraph 3, wherein the immunoglobulin molecule comprises at least one of a V_{H} domain and a V_{L} domain.

5. A compound according to any preceding paragraph, wherein the antigen is specific to a virus.

6. A compound according to paragraph 1, wherein the ligand binds indirectly to the antigen, and is selected from the group consisting of Protein A, Protein G, Protein L, an anti-immunoglobulin peptide and an anti-immunoglobulin antibody.

7. A compound according to any preceding paragraph, wherein the RING domain possesses E3 ligase activity.

8. A compound according to any preceding paragraph, wherein the RING domain is derived from a TRIM polypeptide.

9. A compound according to paragraph 8, wherein the TRIM polypeptide is selected from the group consisting of TRIM5a, TRIM19, TRIM21 and TRIM28.

10. A compound according to any preceding paragraph, which comprises two or more RING domains.

11. A compound according to any preceding paragraph, further comprising a TRIM polypeptide B-box domain and/or a TRIM polypeptide coiled-coil domain.

12. A compound according to paragraph 11, comprising a TRIM polypeptide, wherein the B30.2 domain has been replaced with at least one of a V_{H} domain and a V_{L} domain.

13. A compound according to any one of paragraphs 1 to 12, wherein the inducer of TRIM21 expression is interferon or an interferon inducer.

14. A compound according to paragraph 13, wherein the interferon inducer is selected from the group consisting of a viral or bacterial antigen, a polyanion, a TLR agonist and a small molecule interferon inducer.

15. A compound according to paragraph 13 or paragraph 14, wherein the interferon or interferon inducer is bound to the compound by means of a labile linker.

16. A method for treating a pathogenic infection, comprising administering to a subject a compound according to any preceding paragraph.

17. Use of a compound according to any one of paragraphs 1 to 15, for treating a pathogenic infection.

18. A method for treating an infection in a subject, comprising co-administering to the subject an antibody specific for an antigen of a pathogen causing said infection, and a polypeptide comprising a ligand which binds to said antibody, and a RING domain.

19. Use of an antibody specific for an antigen of a pathogen causing an infection in a subject, and a polypeptide comprising a ligand which binds to said antibody and a RING domain, for the treatment of said infection.

20. A method for treating an infection in a subject suffering from such an infection, comprising administering to the subject a therapeutically effective amount of a polypeptide comprising a polypeptide comprising a ligand which binds, indirectly, to an antigen of a pathogen and a RING domain.

21. Use of a polypeptide comprising a polypeptide comprising a ligand which binds, indirectly, to an antigen of a pathogen and a RING domain for the treatment of an infectious disease in a subject.

22. A method according to paragraph 18 or 20, or the use according to paragraph 19 or 21, wherein the ligand is selected from the group consisting of the TRIM21 PRYSPRY domain, Protein A, Protein G, Protein L, an anti-immunoglobulin peptide and an anti-immunoglobulin antibody.

23. A method or use according to any one of paragraphs 18 to 22, wherein the polypeptide further comprises a TRIM polypeptide coiled coil domain and/or a TRIM polypeptide B-Box domain.

24. A method or use according to paragraph 23, wherein the polypeptide is human TRIM21.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A compound comprising:
(a) a ligand which binds, directly or indirectly, specifically to an antigen of a pathogen, provided that said ligand is not the PRYSPRY domain of TRIM21; and
(b) a RING domain and/or an inducer of TRIM21 expression.

2. A compound according to claim 1, wherein the ligand binds directly to the antigen and is selected from the group consisting of at least part of an immunoglobulin molecule, a peptide and/or nucleic acid aptamer, and a structured polypeptide ligand.

3. A compound according to claim 2, wherein the immunoglobulin molecule is selected from the group consisting of an IgG, IgA, IgM, IgE, IgD, F(ab′)₂, Fab, Fv, scFv, dAb, V_{HH}, IgNAR, a modified TCR, and multivalent combinations thereof.

4. A compound according to claim 3, wherein the immunoglobulin molecule comprises at least one of a V_{H} domain and a V_{L} domain.

5. A compound according to claim 1, wherein the antigen is specific to a virus.

6. A compound according to claim 1, wherein the ligand binds indirectly to the antigen, and is selected from the group consisting of Protein A, Protein G, Protein L, an anti-immunoglobulin peptide and an anti-immunoglobulin antibody.

7. A compound according to claim 1, wherein the RING domain possesses E3 ligase activity.

8. A compound according to claim 1, wherein the RING domain is derived from a TRIM polypeptide.

9. A compound according to claim 8, wherein the TRIM polypeptide is selected from the group consisting of TRIM5a, TRIM19, TRIM21 and TRIM28.

10. A compound according to claim 1, which comprises two or more RING domains.

11. A compound according to claim 1, further comprising a TRIM polypeptide B-box domain and/or a TRIM polypeptide coiled-coil domain.

12. A compound according to claim 11, comprising a TRIM polypeptide, wherein the B30.2 domain has been replaced with at least one of a V_{H} domain and a V_{L} domain.

13. A compound according to claim 1, wherein the inducer of TRIM21 expression is interferon or an interferon inducer.


SEQUENCE LISTING (28 sequences)

SEQ ID NO: 1 - protein, 475 aa, Homo sapiens [sequence omitted]

SEQ ID NO: 2 - DNA, 40 bp, Artificial Sequence
  Description of Artificial Sequence Synthetic VL1S forward primer
acgtgtcgacccaccatggagacagacacactcctgctat

SEQ ID NO: 3 - DNA, 41 bp, Artificial Sequence
  Description of Artificial Sequence Synthetic VL2S forward primer
acgtgtcgacccaccatggattttcaagtgcagattttcag

SEQ ID NO: 4 - DNA, 42 bp, Artificial Sequence
  Description of Artificial Sequence Synthetic VL3S forward primer
acgtgtcgacccaccatggagwcacakwctcaggtctttrta

SEQ ID NO: 5 - DNA, 38 bp, Artificial Sequence
  Description of Artificial Sequence Synthetic VL4S forward primer
acgtgtcgacccaccatgkccccwrctcagytyctkgt

SEQ ID NO: 6 - DNA, 39 bp, Artificial Sequence
  Description of Artificial Sequence Synthetic VL5S forward primer
acgtgtcgacccaccatgaagttgcctgttaggctgttg

SEQ ID NO: 7 - DNA, 33 bp, Artificial Sequence
  Description of Artificial Sequence Synthetic CLX reverse primer
catgtctagactaacactcattcctgttgaagc

SEQ ID NO: 8 - DNA, 42 bp, Artificial Sequence
  Description of Artificial Sequence Synthetic VH1N forward primer
aatagcggccgccaccatggratgsagctgkgtmatsctctt

SEQ ID NO: 9 - DNA, 42 bp, Artificial Sequence
  Description of Artificial Sequence Synthetic VH2N forward primer
aatagcggccgccaccatgracttcgggytgagctkggtttt

SEQ ID NO: 10 - DNA, 41 bp, Artificial Sequence
  Description of Artificial Sequence Synthetic VH3N forward primer
aatagcggccgccaccatggctgtcttggggctgctcttct

SEQ ID NO: 11 - DNA, 43 bp, Artificial Sequence
  Description of Artificial Sequence Synthetic VH4N forward primer
aatagcggccgccaccatgatrgtgttragtcttytgtrcctg

SEQ ID NO: 12 - DNA, 32 bp, Artificial Sequence
  Description of Artificial Sequence Synthetic CHKpn reverse primer
catgggtacctcatttaccaggagagtgggag

SEQ ID NO: 13 - DNA, 1383 bp, Artificial Sequence
  Description of Artificial Sequence Synthetic 9C12 heavy chain polynucleotide
atggctgtcttggggctgctcttctgcctggtgacattcccaagctgtgtcctctcccaggtgcaactgaaacagtcaggacctggcctactgcagccctcacagagactgtccatcacctgcacagtctctggtttctcattgggtcgatatggtgtccactggattcgccagtctcccggaaagggtctggagtggctgggagtgatttggagaggtggaaccacagactataatgcagttttcatgtccagactgagcatcaacaaggacgattccaagagccaagttttctttacaatgaacagtctgcgacctgatgacacagccatatattactgtgccagacagggtagtaacttccctcttgcttactggggccaggggactctggtcactgtctctgccgccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 14 - DNA, 705 bp, Artificial Sequence
  Description of Artificial Sequence Synthetic 9C12 light chain polynucleotide
atggagacacagactcaggtcttcgtatttctactgctctgtgtgtctggtggtcatgggaatattgtgatgacccagactcccaaattcctgcttgtttcaataggagacagtattaccataacctgcaaggccagtcagagtgtgaccaatgatgcagcttggtaccaaaagaagccaggacagtctcctcaactgctgatataccaagcatccactcgctacactggagtccctgatcgcttcagtggcagtggatatgggacggatttcactttcaccatcagcgctgtgcaggctgaagacctggcagtttatttctgtcaccaggattatagctctcctctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 15 - DNA, 1439 bp, Artificial Sequence
  Description of Artificial Sequence Synthetic 9C12 heavy chain, codon optimised polynucleotide
tcggcggccgccaccatggctgtcttggggctgctcttctgcctggtgacattcccaagctgtgtcctctcgagcggatctagacaggtgcaactgaaacagtcaggacctggcctactgcagccctcacagagactgtccatcacctgcacagtctctggtttctcattgggtcgatatggtgtccactggattcgccagtctcccggaaagggtctggagtggctgggagtgatttggagaggtggaaccacagactataatgcagttttcatgtccagactgagcatcaacaaggacgattccaagagccaagttttctttacaatgaacagtctgcgacctgatgacacagccatatattactgtgccagacagggtagtaacttccctcttgcttactggggccaggggactctggtcactgtctctgccgccaaaacgacacccccatctgtctatccactggcccctggatctgctgcccaaactaactccatggtgaccctgggatgcctggtcaagggctatttccctgagccagtgacagtgacctggaactctgggtccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgtatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctctaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaattaattaagtgagtcgacgcaaagatctctcg

SEQ ID NO: 16 - DNA, 752 bp, Artificial Sequence
  Description of Artificial Sequence Synthetic 9C12 light chain, codon optimised polynucleotide
actggtcgacctgcaggccaccatggagacacagactcaggtcttcgtatttctactgctctgtgtgtctggtggtcatgggggtaccgcaatcgataatattgtgatgacccagactcccaaattcctgcttgtttcaataggagacagtattaccataacctgcaaggccagtcagagtgtgaccaatgatgcagcttggtatcaaaagaagccaggacagtctcctcaactgctgatataccaagcatccactcgctacactggagtccctgatcgcttcagtggcagtggatatgggacggatttcactttcaccatcagcgctgtgcaggctgaagacctggcagtttatttctgtcaccaggattatagctctcctctcacgttcggtgctgggaccaagctggagctgaaacgggctgatgctgcaccaactgtatccatcttcccaccatccagtgagcagttgacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttagggatccactg

SEQ ID NO: 17 - protein, 940 aa, Artificial Sequence
  Description of Artificial Sequence Synthetic 9C12-TRIM 21 fusion to CH3 polypeptide
MAVLGLLFCLVTFPSCVLSSGSRQVQLKQSGPGLLQPSQRLSITCTVSGFSLGRYGVHWIRQSPGKGLEWLGVIWRGGTTDYNAVFMSRLSINKDDSKSQVFFTMNSLRPDDTAIYYCARQGSNFPLAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGKLIASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 18 - protein, 700 aa, Artificial Sequence
  Description of Artificial Sequence Synthetic 9C12-RBCC fusion to CH3 polypeptide
MAVLGLLFCLVTFPSCVLSSGSRQVQLKQSGPGLLQPSQRLSITCTVSGFSLGRYGVHWIRQSPGKGLEWLGVIWRGGTTDYNAVFMSRLSINKDDSKSQVFFTMNSLRPDDTAIYYCARQGSNFPLAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGKLIASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRR

SEQ ID NO: 19 - protein, 595 aa, Artificial Sequence
  Description of Artificial Sequence Synthetic 9C12-RB fusion to CH3 polypeptide
MAVLGLLFCLVTFPSCVLSSGSRQVQLKQSGPGLLQPSQRLSITCTVSGFSLGRYGVHWIRQSPGKGLEWLGVIWRGGTTDYNAVFMSRLSINKDDSKSQVFFTMNSLRPDDTAIYYCARQGSNFPLAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGKLIASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEE

SEQ ID NO: 20 - protein, 286 aa, Artificial Sequence
  Description of Artificial Sequence Synthetic protein A-RBCC fusion polypeptide
FNKEQQNAFYEILHLPNLNEEQRNAFIQSLKDDPSQSANLLAEAGSSGSSGMASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRR

SEQ ID NO: 21 - protein, 179 aa, Artificial Sequence
  Description of Artificial Sequence Synthetic protein A-RB fusion polypeptide
FNKEQQNAFYEILHLPNLNEEQRNAFIQSLKDDPSQSANLLAEAGSSGSSGMASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPL

SEQ ID NO: 22 - protein, 119 aa, Artificial Sequence
  Description of Artificial Sequence Synthetic protein A-R fusion polypeptide
FNKEQQNAFYEILHLPNLNEEQRNAFIQSLKDDPSQSANLLAEAGSSGSSGMASAARLTMMWEEVTCPICLDPFVEPVSIECGHSFCQECISQVGKGGGSVCPVCRQRFLLKNLRPNRQ

SEQ ID NO: 23 - protein, 337 aa, Artificial Sequence
  Description of Artificial Sequence Synthetic RBCC-protein A fusion polypeptide
MASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPGLKKMLRTCAFNKEQQNAFYEILHLPNLNEEQRNAFIQSLKDDPSQSANLLAEAGSSGSSG

SEQ ID NO: 24 - protein, 107 aa, Artificial Sequence
  Description of Artificial Sequence Synthetic ′Cross-brace′ motif polypeptide
CXXCXXXXXXXXXXXXXXXXXXXXXXXXXXXXXXXXXXXXXXXCXXXHXXXXXXCXXXXXXXXXXXXXXXXXXXXXXXXXXXXXXXXXXXXXXXXXXXXXXXXCXXC

SEQ ID NO: 25 - DNA, 19 bp, Homo sapiens
gcagcacgcttgacaatga

SEQ ID NO: 26 - RNA, 19 nt, Artificial Sequence
  Description of Artificial Sequence Synthetic small interfering (si) RNA T21siRNA1 oligonucleotide
ucauugucaagcgugcugc

SEQ ID NO: 27 - RNA, 24 nt, Artificial Sequence
  Description of Artificial Sequence Synthetic small interfering (si) RNA T21siRNA2 oligonucleotide
uggcauggaggcaccugaaggugg

SEQ ID NO: 28 - protein, 6 aa, Artificial Sequence
  Description of Artificial Sequence Synthetic 6xHis tag
HHHHHH